(19) United States
(12) Patent Application Publication
(10) Pub. No.: US 2025/0040863 A1
(43) Pub. Date: Feb. 6, 2025

(54) SYSTEMS AND METHODS FOR GENERATING SPATIOTEMPORAL SENSORY CODES

(71) Applicant: DANDELION SCIENCE CORP., New York, NY (US)

(72) Inventors: Adam HANINA, New York, NY (US); Ekaterina MALAKHOVA, Highlands Mills, NY (US); Dan NEMRODOV, Jersey City, NJ (US); David KLEIN, Los Altos, CA (US)

(21) Appl. No.: 18/696,268

(22) PCT Filed: Sep. 28, 2022
(86) PCT No.: PCT/US2022/077207

§ 371 Date: Mar. 27, 2024

Related U.S. Application Data
(60) Provisional application No. 63/249,314, filed on Sep. 28, 2021.

(51) Int. Cl. A61B 5/378; A61B 5/00; G16H 20/30

(52) U.S. Cl. CPC A61B 5/378; A61B 5/0042; G16H 20/30

(57) ABSTRACT

Systems and methods for providing spatiotemporal sensory inputs to participants to produce a stimulus map of the brain. A code generation model is sampled with a first encoding vector to produce a first video sequence, which is output to provide a first spatiotemporal sensory input to the participants. Neural response measurements are performed while the first spatiotemporal sensory input is being presented to each of the participants. An outcome function is determined based on the neural response measurements. A second encoding vector is produced based on the first encoding vector and the outcome function. The method is iteratively repeated with the second encoding vector, and any successive encoding vectors, until a defined set of stopping criteria for the outcome function is satisfied. Upon satisfying the defined set of stopping criteria, a resulting spatiotemporal sensory code is stored to form part of a stimulus map of the brain.

### CROSS REFERENCE TO RELATED APPLICATIONS

The present application claims priority to U.S. Provisional Patent Application No. 63/249,314, filed Sep. 28, 2021, which is hereby incorporated by reference in its entirety.

### BACKGROUND

### Technical Field

The present disclosure generally relates to generating spatiotemporal sensory codes using computer vision techniques to produce a stimulus map of the brain.

### Description of the Related Art

Neurons in the visual cortex fire action potentials when visual stimuli, e.g., images, appear within their receptive field. By definition, the receptive field is the region within the entire visual field that elicits an action potential. But, for any given neuron, it may respond best to a subset of stimuli within its receptive field. This property is called neuronal tuning. In the earlier visual areas, neurons have simpler tuning. For example, a neuron in V1 may fire to any vertical stimulus in its receptive field. In the higher visual areas, neurons have complex tuning. For example, in the inferior temporal cortex (IT), a neuron may fire only when a certain face appears in its receptive field.

A challenge in delineating neuronal tuning in the visual cortex is the difficulty of selecting particular stimuli from the vast set of all possible stimuli. Using natural images reduces the problem, but it is impossible to present a neuron with all possible natural stimuli. Conventionally, investigators have used hand-picked stimuli based on hypotheses that particular cortical areas encode specific visual features. Despite some success with hand-picked stimuli, the field might have missed stimulus properties that better reflect the potential of tuning of cortical neurons.

Current clinical approaches to the diagnosis of neurofunctional and psychiatric disorders rely heavily on behavioral symptoms and subjective reports, and inferential diagnostic tools. Diagnostic imaging and biometric tools are unable to detect pathology with enough specificity, which reduces the medical art to a somewhat crude diagnosis of exclusion rather than truly diagnostic. For example, in the case of neurological transmission disruption, there is a lack of effective biomapping tools to objectively demonstrate functional disturbances, e.g., demyelination, degeneration, and disruption.

Within central nervous system drug development, clinical trials are slow and expensive. The rate of new approvals is markedly lower than for other therapeutic areas. Progress is hindered by poor target validation, low specificity, absence of biomarkers, and difficulty in replicating trial results in real-world settings, especially in heterogeneous populations.

### SUMMARY

Disclosed embodiments provide a spatiotemporal biomapping platform that enables an objective performance-based description of neurofunctional and psychiatric disorders. In embodiments, the platform has: (a) broad network and neural coverage resulting from non-invasive visual and/or audio tests; (b) the ability to create new indication-specific maps quickly and robustly and then apply that knowledge on an individual patient level; and (c) the ability to generate inference metrics that provide generalizable, reliable, and standardized insights about neural information processing useful for various stages of the development and delivery of therapies.

Visual pathways activated by dynamic visual stimuli have the potential to engage a significant area of the cortex and act as a functional diagnostic tool, thus providing an understanding of associated dysfunctional circuitry. Once the circuitry is understood, the opportunity arises to develop therapeutic neuromodulatory effects by means of a sequence of visual stimuli.

Disclosed embodiments provide a therapeutic platform with neuromodulatory stimuli based on illness “circuits” and pathways defined using the methods described herein. These approaches provide, in effect, a targeted neuromodulatory language, which facilitates precision therapeutic stimuli. In disclosed embodiments, the brain mapping and therapeutic objectives are facilitated by the disclosed systems and methods for predicting target pathways via closed loop neurostimulation using complex spatiotemporal visual stimuli that optimize in-loop.

Disclosed embodiments provide a therapeutic-discovery platform capable of generating sensory stimuli, e.g., visual and/or audial stimuli, for a wide range of disorders. Dynamic visual neuromodulatory codes are viewed, e.g., on the screen of a laptop, smartphone, or VR headset, when a patient experiences symptoms. Designed to be inexpensive, noninvasive, and convenient to use, the sensory codes offer immediate and potentially sustained relief without requiring clinician interaction. Sensory codes are being developed for, inter alia, acute pain, fatigue and acute anxiety, thereby broadening potential treatment access for many who suffer pain or anxiety.

Disclosed embodiments involve the use of non-figurative (i.e., abstract, non-semantic, and/or non-representational) visual stimuli, such as the visual neuromodulatory codes described herein, which have advantages over figurative content. Non-figurative visual stimuli can be brought under tight experimental control for the purpose of stimulus optimization. Under AI guidance, specific features (e.g., shape, color, duration, movement, frequency, hue, etc.) can be expressed as parameters and gradually readjusted and recombined, frame by frame, pixel by pixel, to drive bioresponse in the desired direction. Unlike pictures of people or scenes, non-figurative visual stimuli are free of cultural or language bias and thus more generalizable as a global therapeutic.

To activate specific targeted areas in the visual cortex, neuronal selectivity can be examined using the vast hypothesis space of a generative deep neural network, without assumptions about features or semantic categories. A genetic algorithm can be used to search this space for stimuli that maximize neuronal firing and/or feedback data indicative of responses of a user, or group of participants, during display of the stimuli. This allows for the evolution of synthetic images of objects with complex combinations of shapes, colors, and textures, sometimes resembling animals or familiar people, other times revealing novel patterns that do not map to any clear semantic category.

In disclosed embodiments, a combination of a pre-trained deep generative neural network and a genetic algorithm can be used to allow neuronal responses and/or feedback data indicative of responses of a user, or group of participants, during display of the stimuli to guide the evolution of synthetic images. By training large numbers of images, a generative adversarial network can learn to model the statistics of natural images without merely memorizing the training set, thus representing a vast and general image space constrained only by natural image statistics. This provides an efficient space in which to perform a genetic algorithm, because the brain also learns from real-world images, so its preferred images are also likely to follow natural image statistics.

Disclosed embodiments may include an end-to-end computer vision platform. Visual stimuli, e.g., visual neuromodulatory codes, are created by computational graphics and then characterized, i.e., parameterized, using computer vision techniques. In such an approach, the complexity of the graphics being created can be described in highly measurable manner, which allows for description of, for example, movement, shape formation, the complexity of a number of items occurring on a display screen at any one time (e.g., arrangements of items). These aspects, inter alia, can be described by computer vision by creating sophisticated computer vision descriptors. Moreover, in this manner, the graphics (e.g., visual neuromodulatory codes) are parameterized, which allows for control of the creation and presentation of the computational graphics and, thus, the input of the end-to-end computer vision platform.

Disclosed embodiments provide dynamic neural responses, using visual neuromodulatory images or codes, in a predictable and reliable manner. In this approach, a mapping is developed between visual neuromodulatory images or codes and the dynamic neural responses. From the mapping, one can infer characteristics of the brain which are analogous to parameters used to characterize data networks, such as, for example, as processing speed, bandwidth, network connectivity and efficiency, and, furthermore, brain characteristics relating to the ability to solve problems. The mapping, and the inferences drawn from it, can be used to characterize a spectrum of performance based on measurements from a number of different individuals. This, in turn, allows for a sort of phenotyping of patient populations for the support of both diagnosis and drug development.

In one aspect, the disclosed embodiments provide a method for providing spatiotemporal sensory inputs to one or more participants to produce a stimulus map of the brain. The method includes sampling a spatiotemporal sensory code generation model with a first encoding vector to produce a first spatiotemporal sensory code in the form of a first video sequence. The method further includes outputting the first video sequence to provide a first spatiotemporal sensory input to said one or more participants. The method further includes receiving one or more neural response measurements for said one or more participants, said one or more neural response measurements being performed while the first spatiotemporal sensory input is being presented to each respective one of said one or more participants. The method further includes determining an outcome function based, at least in part, on said one or more neural response measurements for said one or more participants. The method further includes producing a second encoding vector based, at least in part, on the first encoding vector and the outcome function. The method further includes iteratively repeating said sampling, said outputting, said receiving, and said determining with the second encoding vector, and any successive encoding vectors, until a defined set of stopping criteria for the outcome function is satisfied. Upon satisfying the defined set of stopping criteria for the outcome function, a resulting spatiotemporal sensory code is stored to form part of a stimulus map of the brain.

Embodiments may include one or more of the following features, separately or in any feasible combination.

The spatiotemporal sensory codes may include one or more of the following: visual sensory inputs, auditory sensory inputs, and somatosensory inputs. The generation model may include procedural graphics using input parameters including one or more of: spatial frequencies, temporal frequencies, spatial locations, spatial extents, and translation-based motion vectors. The spatiotemporal sensory code generation model may include a generative adversarial network or deep diffusion model and the first encoding vector may point to a location in a latent generation space.

The spatiotemporal sensory codes, in the form of video sequences, have a defined time length and partially overlap in time. The first video sequence may have N frames starting from time Tᵢ, and the method may further include: applying a per-frame window function to the first video sequence; and adding the result to an output frame buffer, filling frames from Tᵢ to Tᵢ+N. The successive encoding vectors may be produced based at least in part on the outcome function and a plurality of preceding encoding vectors.

The producing of the second encoding vector may be done at time Tᵢ+S, where S<=N, and the method may further include: applying the per-frame window function to the second video sequence; and adding the result to the output frame buffer, resulting in the output frame buffer comprising frames Tᵢ to Tᵢ+S+N. During said outputting, frames from Tᵢ to Tᵢ+S may be output from the output frame buffer to be presented to said one or more participants while the second video sequence is being produced.

The outputting may include displaying said sequence of spatiotemporal sensory inputs to one or more electronic screens. The one or more neural response measurements may be performed using one or more of the following: electroencephalogram (EEG), quantitative EEG, magnetoencephalography (MEG), single-photon emission computed tomography (SPECT), positron emission tomography (PET), functional magnetic resonance imaging (fMRI), functional near-infrared spectroscopy (fNIRS), electrocardiogram (EKG) measurement data, pulse rate data, galvanic skin response, and blood pressure data. The one or more neural response measurements may be received from a multiple-channel buffer including current multiple-channel neural response measurements and previous multiple-channel neural response measurements.

The method may further include aligning timewise, across said one or more participants, said one or more neural response measurements; extracting one or more features for each measurement time step across said one or more neural response measurements and across said one or more participants; and comparing said one or more extracted features to targets to calculate the outcome function. The defined set of stopping criteria may include one or more of the following: specified convergence criteria, a specified number of iterations, and a specified amount of time.

In storing said resulting spatiotemporal sensory code to form part of the stimulus map of the brain, a feature representation of said one or more neural response measurements may be associated with a location in a high dimensional space. The resulting spatiotemporal sensory code may be associated with a neural state at a specific brain location. The resulting spatiotemporal sensory code may be associated with a whole-brain neural state. The whole-brain neural state may be defined in terms of multivariate cross-coherence across spectral bands and said resulting spatiotemporal sensory code may be adapted to maximize the cross-coherence across one or more pairs of nodes of the brain map.

In another aspect, the disclosed embodiments provide a system for providing spatiotemporal sensory inputs to one or more participants to produce a stimulus map of the brain. The system includes at least one processor; and at least one non-transitory processor-readable medium that stores processor-executable instructions which, when executed by said at least one processor, cause the at least one processor to perform the methods discussed above.

### BRIEF DESCRIPTION OF THE DRAWINGS

FIG. 1 depicts an embodiment of a system to generate and optimize non-figurative visual neuromodulatory codes implemented using an “inner loop” which optimizes visual neuromodulatory codes through biomedical sensor feedback to maximize the therapeutic impact for an individual subject or group of subjects and an “outer loop” which uses various processing techniques to generalize the effectiveness of the visual neuromodulatory codes produced by the inner loop for the general population of users.

FIG. 2 depicts an embodiment of a system to generate non-figurative visual neuromodulatory codes adapted to produce physiological responses having therapeutic or performance-enhancing effects.

FIG. 3 depicts an embodiment of a method, usable with the system of FIG. 2, to generate visual neuromodulatory codes to produce physiological responses having therapeutic or performance-enhancing effects.

FIG. 4 depicts an embodiment of a method, usable with the system of FIG. 18, to provide visual neuromodulatory codes to produce physiological responses having therapeutic or performance-enhancing effects.

FIG. 5 depicts an embodiment of a system to generate and provide to a user a visual stimulus, using visual codes displayed to a group of participants, to produce physiological responses having therapeutic or performance-enhancing effects.

FIG. 6 depicts an embodiment of a method, usable with the system of FIG. 5, to generate a visual stimulus to produce physiological responses having therapeutic or performance-enhancing effects.

FIG. 7 depicts an initial population of images created from random achromatic textures constructed from a set of textures which are derived from randomly sampled photographs of natural objects on a gray background.

FIG. 8 depicts an embodiment of a system to generate a visual stimulus, using brain state data and/or brain activity data measured while visual codes are displayed to a participant in a target state and a current state, to produce physiological responses having therapeutic or performance-enhancing effects.

FIG. 9 depicts an embodiment of a method, usable with the system of FIG. 8, to generate a visual stimulus to produce physiological responses having therapeutic or performance-enhancing effects.

FIG. 10 depicts an embodiment of a system to deliver a visual stimulus, generated using visual codes displayed to a group of participants, to produce physiological and/or neurological responses.

FIG. 11 depicts an embodiment of a method to deliver a visual stimulus, usable with the system of FIG. 10.

FIG. 12 depicts a method for providing spatiotemporal sensory inputs to one or more participants to produce a stimulus map of the brain.

FIG. 13 depicts an embodiment of a system to deliver a visual stimulus, generated using brain state data and/or brain activity data measured while visual codes are displayed to a participant in a target state and a current state, to produce physiological responses having therapeutic or performance-enhancing effects.

FIG. 14 depicts an embodiment of a method to deliver a visual stimulus, usable with the system of FIG. 13, to produce physiological responses having therapeutic or performance-enhancing effects.

FIG. 15 depicts an embodiment of a system to generate visual neuromodulatory codes with a closed-loop approach using an optimized descriptive space.

FIG. 16 depicts an embodiment of a method, usable with the system of FIG. 15, to generate visual neuromodulatory codes with a closed-loop approach using an optimized descriptive space.

FIG. 17 depicts an embodiment of a method to determine an optimized descriptive space to characterize visual neuromodulatory codes.

FIG. 18 depicts an embodiment of a system to deliver visual neuromodulatory codes generated with a closed-loop approach using an optimized descriptive space.

FIG. 19 depicts an embodiment of a method, usable with the system of FIG. 18, to deliver visual neuromodulatory codes generated with a closed-loop approach using an optimized descriptive space according to the method of FIG. 16.

FIG. 20 depicts an embodiment of a system to generate visual neuromodulatory codes by reverse correlation and stimuli classification.

FIG. 21 depicts an embodiment of a method, usable with the system of FIG. 20 to generate visual neuromodulatory codes by reverse correlation and stimuli classification.

FIG. 22 depicts an embodiment of a method, usable with the system of FIG. 18, to deliver visual neuromodulatory codes generated by reverse correlation and stimuli classification according to the method of FIG. 21.

### DETAILED DESCRIPTION

In the following description, certain specific details are set forth in order to provide a thorough understanding of various disclosed implementations. However, one skilled in the relevant art will recognize that implementations may be practiced without one or more of these specific details, or with other methods, components, materials, etc. In other instances, well-known structures associated with computer systems, server computers, and/or communications networks have not been shown or described in detail to avoid unnecessarily obscuring descriptions of the implementations.

Unless the context requires otherwise, throughout the specification and claims that follow, the word “comprising” is synonymous with “including,” and is inclusive or open-ended (i.e., does not exclude additional, unrecited elements or method acts). Reference throughout this specification to “one implementation” or “an implementation” or “particular implementations” means that a particular feature, structure or characteristic described in connection with the implementation is included in at least one implementation. Thus, the appearances of the phrases “in one implementation” or “in an implementation” or “particular implementations” in various places throughout this specification are not necessarily all referring to the same implementation. Furthermore, the particular features, structures, or characteristics may be combined in any suitable manner in one or more implementations.

As used in this specification and the appended claims, the singular forms “a,” “an,” and “the” include plural referents unless the context clearly dictates otherwise. It should also be noted that the term “or” is generally employed in its sense including “and/or” unless the context clearly dictates otherwise. The headings and Abstract of the Disclosure provided herein are for convenience only and do not interpret the scope or meaning of the implementations.

Physiology is a branch of biology that deals with the functions and activities of life or of living matter (e.g., organs, tissues, or cells) and of the physical and chemical phenomena involved. It includes the various organic processes and phenomena of an organism and any of its parts and any particular bodily process. Hence, the term “physiological” is used herein to broadly mean characteristic of or appropriate to the functioning of an organism, including human physiology. The term includes the characteristics and functioning of the nervous system, the brain, and all other bodily functions and systems.

The term “neurophysiology” refers to the physiology of the nervous system. The term “neural” and the prefix “neuro” likewise refer to the nervous system. As used herein, all of these terms and prefixes refer to the physiology of the nervous system and brain. In some instances, these terms and prefixes are used herein to refer to physiology more generally, including the nervous system, the brain, and physiological systems which are physically and functionally related to the nervous system and the brain.

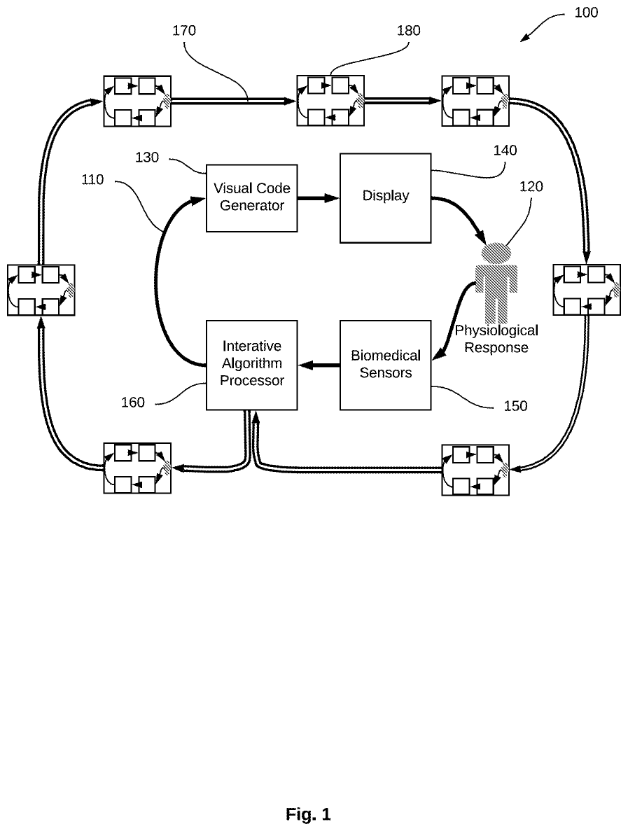

FIG. 1 depicts an embodiment of a system 100 to generate and optimize visual neuromodulatory codes to produce physiological responses having therapeutic or performance-enhancing effects. The system 100 combines visual synthesis technologies, real-time physiological feedback (including neurofeedback) processing, and artificial intelligence guidance to generate stimulation parameters to accelerate discovery and optimize therapeutic effect of visual neuromodulatory codes. The system is implemented in two stages: an “inner loop” which optimizes visual neuromodulatory codes through biomedical sensor feedback to maximize the therapeutic impact for an individual subject or group of subjects; and an “outer loop” which uses various processing techniques to generalize the effectiveness of the visual neuromodulatory codes produced by the inner loop for the general population of users. It should be noted that although the phrase “therapeutic or performance-enhancing effects” is used throughout the present application, in some cases an effect may have both a therapeutic and a performance-enhancing aspect, so it should be understood that physiological responses may have therapeutic or performance-enhancing effects or both. The term “performance-enhancing” refers to effects such as stimulation (i.e., as with caffeine), improved focus, improved attention, etc.

In embodiments, to maximize the chances of discovering responses that are consistent across subjects, optimization may be carried out on a group basis, in which case a group of subjects is presented simultaneously with visual images in the form of visual neuromodulatory codes. The bio-responses of the group of subjects are aggregated and analyzed in real time to determine which stimulation parameters (i.e., the parameters used to generate the visual neuromodulatory codes) are associated with the greatest response. The system optimizes the stimuli, readjusting and recombining the visual parameters to quickly drive the collective response of the group of subjects in the direction of greater response. Such group optimization increases the chances of evoking ranges of finely graded responses that have cross-subject consistency.

The system 100 includes an iterative inner loop 110 which synthesizes and refines visual neuromodulatory codes based on the physiological responses of an individual subject (e.g., 120) or group of subjects. The inner loop 110 can be implemented as specialized equipment, e.g., in a facility or laboratory setting, dedicated to generating therapeutic visual neuromodulatory codes. Alternatively, or in addition, the inner loop 110 can be implemented as a component of equipment used to deliver therapeutic visual neuromodulatory codes to users, in which case the subject 120 (or subjects) is also a user of the system.

The inner loop 110 includes a visual stimulus generator 130 to synthesize visual neuromodulatory codes, which may be in the form of a set of one or more visual neuromodulatory codes defined by a set of image parameters (e.g., “rendering parameters”). In implementations, the synthesis of the visual neuromodulatory codes may be based on artificial intelligence-based manipulation of image data and image parameters. The visual neuromodulatory codes are output by the visual stimulus generator 130 to a display 140 to be viewed by the subject 120 (or subjects). Physiological responses of the subject 120 (or subjects) are measured by biomedical sensors 150, e.g., electroencephalogram (EEG), pulse rate, and blood pressure, while the visual neuromodulatory codes are being presented to the subject 120 (or subjects).

The measured physiological data is received by an iterative algorithm processor 160, which determines whether the physiological responses of the subject 120 (or subjects) meet a set of target criteria. If the physiological responses of the subject 120 (or subjects) do not meet the target criteria, then a set of adapted image parameters is generated by the iterative algorithm processor 160 based on the output of the sensors 150. The adapted image parameters are used by the visual stimulus generator 130 to produce adapted visual neuromodulatory codes to be output to the display 140. The iterative inner loop process continues until the physiological responses of the subject 120 (or subjects) meet the target criteria, at which point the visual neuromodulatory codes have been optimized for the particular subject 120 (or subjects).

An “outer loop” 170 of the system 100 provides for the generalization of visual neuromodulatory codes from a wide-ranging population of subjects and/or users. In the generalization process, optimized image parameters from a number of instances of inner loops 180 are processed to produce a generalized set of image parameters which have a high likelihood of being effective for a large number of users. The generalized set of image parameters evolves over time as additional subjects and/or users are included in the outer loop 170. As more patients use the system 100, the outer loop uses techniques such as ensemble and transfer learning to distill visual neuromodulatory codes into “dataceuticals” and optimize their effects to be generalizable across patients and conditions. By encoding visual information in a manner similar to the visual cortex through the use of artificial intelligence, visual neuromodulatory codes can efficiently activate brain circuits and expedite the search for optimal stimulation, thereby creating, in effect, a visual language for interfacing with and healing the brain.

Among the advantages of the system 100 is that it effectively accelerates central nervous system (CNS) translational science, because it allows therapeutic hypotheses to be tested quickly and repeatedly through artificial intelligence-guided iterations, thereby significantly speeding up treatment discovery by potentially orders of magnitude and increasing the chances of providing relief to millions of untreated and undertreated people worldwide.

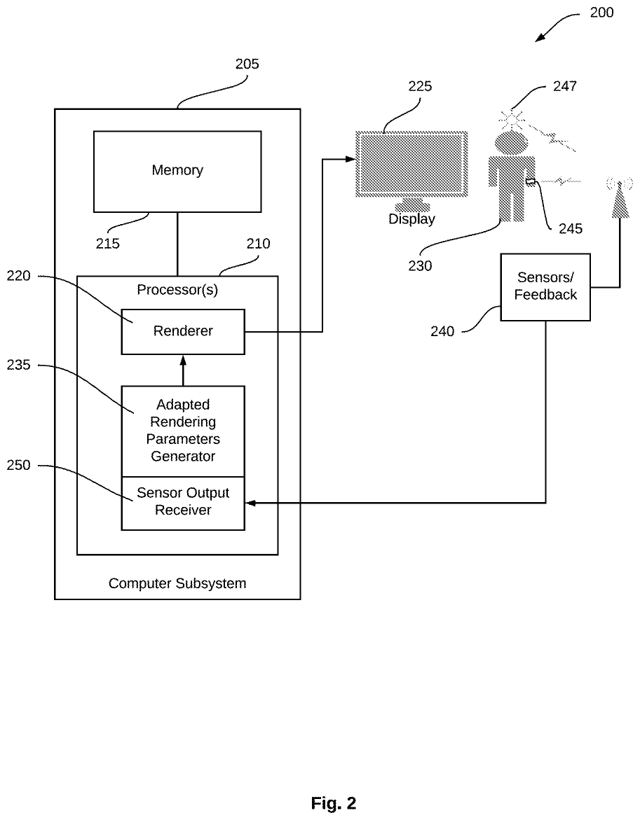

FIG. 2 depicts an embodiment of a system 200 to generate visual neuromodulatory codes to produce physiological responses having therapeutic or performance-enhancing effects (or both). The system 200 includes a computer subsystem 205 comprising at least one processor 210 and memory 215 (e.g., non-transitory processor-readable medium). The memory 215 stores processor-executable instructions which, when executed by the at least one processor 210, cause the at least one processor 210 to perform a method to generate the visual neuromodulatory codes. Specific aspects of the method performed by the processor 210 are depicted as elements (e.g., code, software modules, and/or processes) within the processor for purposes of discussion only.

The renderer 220 performs a rendering process to produce images (e.g., sequences of images) to be displayed on the display 225 by generating video data based on specific inputs. In implementations, the output of the rendering process is a digital image stored as an array of pixels. Each pixel value may be a single scalar component or a vector containing a separate scalar value for each color component. The renderer 220 may produce (i.e., synthesize) one or more visual neuromodulatory codes (e.g., a sequence of visual neuromodulatory codes) based on an initial set of rendering parameters (i.e., synthesis parameters) stored in the memory 215. The video data and/or signal resulting from the rendering is output by the computer subsystem 205 to the display 225.

The system 200 is configured to output the visual neuromodulatory codes to a display 225 viewable by a subject 230 or a number of subjects simultaneously. For example, a video monitor may be provided in a location where it can be accessed by the subject 230 (or subjects), e.g., a location where other components of the system are located. Alternatively, the video data may be transmitted via a network to be displayed on a video monitor or mobile device (not shown) of the subject (or subjects). In implementations, the subject 230 (or subjects) may be one of the users of the system.

In implementations, the system 200 may output to the display 225 a dynamic visual neuromodulatory code based on a plurality of visual neuromodulatory codes. For example, a dynamic visual neuromodulatory code may be formed by combining a number of visual neuromodulatory codes to form a sequence of visual neuromodulatory codes. In a further example, a dynamic visual neuromodulatory code may be adapted to produce at least one of the following effects: a pulsating effect, a zooming effect, a flickering effect, and a color-shift effect. In some cases, the formation of the dynamic visual neuromodulatory code may include processing a set, e.g., a sequence, of visual neuromodulatory codes to produce intermediate images in the sequence of visual neuromodulatory codes. Various techniques, such as interpolation of pixels and gaussian averaging, may be used to produce the intermediate images.

The system 200 includes one or more sensors 240, such as biomedical sensors, to measure physiological responses of the subject 230 (or subjects) while the visual neuromodulatory codes are being presented to the subject 230 (or subjects). For example, the system may include a wristband 245 and a head-worn apparatus 247 and may also include various other types of physiological and neurological feedback devices. In general, biomedical sensors include physical sensors, chemical sensors, and biological sensors. Physical sensors may be used to measure and monitor physiologic properties such as, for example, physical blood pressure, respiration, pulse, body temperature, heart sound, respiratory rate, blood viscosity, flow rate, flow rate, etc. Chemical sensors may be utilized to measure chemical parameters, such as, for example, oxygen and carbon dioxide concentration in the human metabolism, pH value, and ion levels in bodily fluids. Biological sensors (i.e., “biosensors”) are used to detect biological parameters, such as tissues, cells, enzymes, antigens, antibodies, receptors, hormones, cholic acid, acetylcholine, serotonin, DNA and RNA, and other proteins and biomarkers.

The sensors 240 used in the system 200 may include wearable devices, such as, for example, wristbands 245 and head-worn apparatuses 247. Other examples of wearable devices include smart glasses, watches, fitness bands/watches, running shoes, rings, armbands, belts, helmets, buttons, etc. In implementations, the physiological responses of the subject 230 (or subjects) may be measured using sensors adapted to measure, inter alia, one of the following: neurological responses, physiological responses, and behavioral responses. The sensors 240 may include one or more of the following: EEG, MEG, fMRI, ECG, EMG, electrocardiogram, pulse rate, and blood pressure. In some cases, wearable devices may identify a specific neural state, e.g., an epilepsy kindling event, thereby allowing the system to respond to counteract the state, artificial intelligence-guided visual neuromodulatory codes can be presented to counteract and neutralize the kindling with high specificity.

A sensor output receiver 250 of the computer subsystem 205 receives the outputs of the sensors 240, e.g., data and/or analog electrical signals, which are indicative of the physiological responses of the subject 230 (or subjects), as measured by the sensors 240 during the output of the visual neuromodulatory codes to the display 225. In implementations, the analog electrical signals may be converted into data by an external component, e.g., an analog-to-digital converter (ADC) (not shown). Alternatively, the computer subsystem 205 may have an internal component, e.g., an ADC card, installed to directly receive the analog electrical signals. Data output received from the sensors 240 in various forms and protocols, such as via a serial data bus or via network protocols, e.g., UDP or TCP/IP. The sensor output receiver 250 converts the sensor outputs, as necessary, into a form usable by the adapted rendering parameter generator 235.

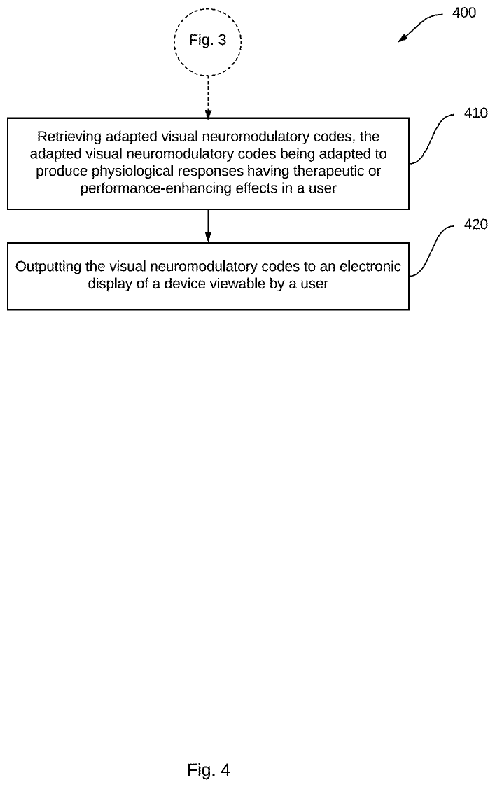

If measured physiological responses of the subject 230 (or subjects) do not meet a set of target criteria, the adapted rendering parameter generator 235 generates a set of adapted rendering parameters based at least in part on the received output of the sensors. The adapted rendering parameters are passed to the renderer 220 to be output to the display 225, as described above. The system 200 iteratively repeats the rendering (e.g., by the renderer 220), outputting the visual neuromodulatory codes to a display 225 viewable by the subject 230 (or subjects), and the receiving output of sensors 240 that measure, during the outputting of the visual neuromodulatory codes to the display 225, the physiological responses of the subject 230 using the adapted rendering parameters. The iterations are performed until the physiological responses of the subject 230 (or subjects), as measured by the sensors 240, meet the target criteria, at which point the system 200 outputs the visual neuromodulatory codes to be used in producing physiological responses having therapeutic or performance-enhancing effects (or both). In implementations, the adapted visual neuromodulatory codes may be used in a method to provide visual neuromodulatory codes (see, e.g., FIG. 4 and related description below).

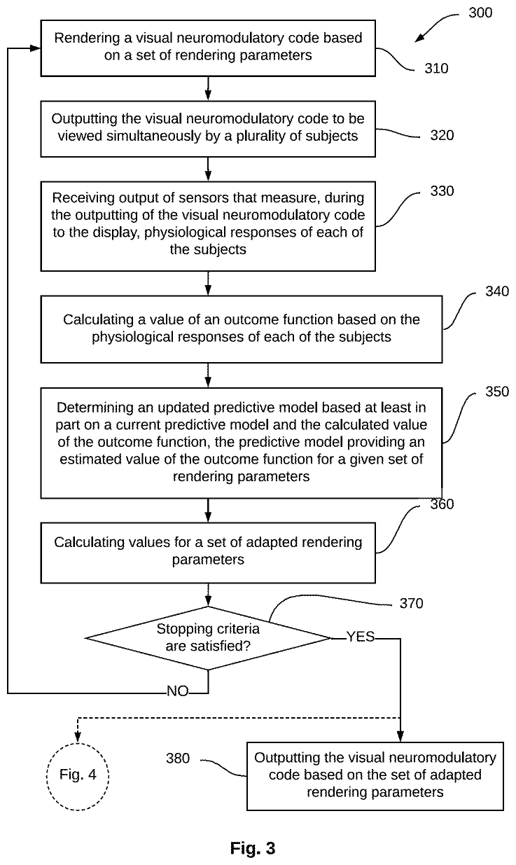

FIG. 3 depicts an embodiment of a method 300, usable with the system of FIG. 2, to generate visual neuromodulatory codes to produce physiological responses having therapeutic or performance-enhancing effects (or both).

In embodiments, a Bayesian optimization may be performed to adapt the rendering parameters—and hence optimize the resulting visual neuromodulatory codes—based on the physiological responses of the subjects. In particular, the optimization aims to drive the physiological responses of the subjects based on target criteria, which may be a combination of thresholds and/or ranges for various physiological measurements performed by sensors. For example, to achieve a therapeutic response which reduces stress, target criteria may be established which are indicative of a reduction in pulse rate and/or blood pressure. Using such an approach, the method can efficiently search through a large experiment space (e.g., the set of all possible rendering parameters) with the aim of identifying the experimental condition (e.g., a particular set of rendering parameters) that exhibits an optimal response in terms of physiological responses of subjects. In some embodiments, other analysis techniques, such as dynamic Bayesian networks, temporal event networks, and temporal nodes Bayesian networks, may be used to perform all or part of the adaptation of the rendering parameters.

The relationship between the experiment space and the physiological responses of the subjects may be quantified by an objective function (or “cost function”), which may be thought of as a “black box” function. The objective function may be relatively easy to specify but can be computationally challenging to calculate or result in a noisy calculation of cost over time. The form of the objective function is unknown and is often highly multi-dimensional depending on the number of input variables. For example, a set of rendering parameters used as input variables may include a multitude of parameters which characterize a rendered image, such as shape, color, duration, movement, frequency, hue, etc. In the example mentioned above, in which the goal is to achieve a therapeutic response which reduces stress, the objective function may be expressed in terms of neurophysiological features calculated from rate and/or blood pressure, e.g., heart rate variability and ratio systolic and diastolic blood pressure, each multiplied by scaling coefficients. In some embodiments, only a single physiological response may be taken into account by the objective function.

The optimization involves building a probabilistic model (referred to as the “surrogate function” or “predictive model”) of the objective function. The predictive model is progressively updated and refined in a closed loop by automatically selecting points to sample (e.g., selecting particular sets of rendering parameters) in the experiment space. An “acquisition function” is applied to the predictive model to optimally choose candidate samples (e.g., sets of rendering parameters) for evaluation with the objective function, i.e., evaluation by taking actual sensor measurements. Examples of acquisition functions include probability of improvement (PI), expected improvement (EI), and lower confidence bound (LCB).

The method 300 includes rendering a visual neuromodulatory code based on a set of rendering parameters (310). Various types of rendering engines may be used to produce the visual neuromodulatory code (i.e., image), such as, for example, procedural graphics, generative neural networks, gaming engines and virtual environments. Conventional rendering involves generating an image from a 2D or 3D model. Multiple models can be defined in a data file containing a number of “objects,” e.g., geometric shapes, in a defined language or data structure. A rendering data file may contain parameters and data structures defining geometry, viewpoint, texture, lighting, and shading information describing a virtual “scene.” While some aspects of rendering are more applicable to figurative images, i.e., scenes, the rendering parameters used to control these aspects may nevertheless be used in producing abstract, non-representational, and/or non-figurative images. Therefore, as used herein, the term “rendering parameter” is meant to include all parameters and data used in the rendering process, such that a rendered image (i.e., the image which serves as the visual neuromodulatory code) is completely specified by its corresponding rendering parameters.

In some embodiments, the rendering of the visual neuromodulatory code based on the set of rendering parameters may include projecting a latent representation of the visual neuromodulatory code onto the parameter space of a rendering engine. Depending on the rendering engine, the final appearance of the visual neuromodulatory code may vary, however the desired therapeutic properties are preserved.

The method further includes outputting the visual neuromodulatory code to be viewed simultaneously by a plurality of subjects (320). The method 300 further includes receiving output of one or more sensors that measure, during the outputting of the visual neuromodulatory code, one or more physiological responses of each of the plurality of subjects (330).

The method 300 further includes calculating a value of an outcome function based on the physiological responses of each of the plurality of subjects (340). The outcome function may act as a cost function (or loss function) to “score” the sensor measurements relative to target criteria. the outcome function is indicative of a therapeutic effectiveness of the visual neuromodulatory code.

The method 300 further includes determining an updated predictive model based at least in part on a current predictive model and the calculated value of the outcome function—the predictive model providing estimated value of the outcome function for a given set of rendering parameters (350).

The method 300 further includes calculating values for a set of adapted rendering parameters (360). The values may be calculated based at least in part on determining, using the updated predictive model, an estimated value of the outcome function for a plurality of values of the set of rendering parameters to form a response characteristic (e.g., response surface); and determining values of the set of adapted rendering parameters based at least in part on the response characteristic. In some embodiments, an acquisition function may be applied to the response characteristic to optimize selection of the values of the set of adapted rendering parameters.

The method 300 is iteratively repeated using the adapted rendering parameters until a defined set of stopping criteria are satisfied (370). Upon satisfying the defined set of stopping criteria, the visual neuromodulatory code based on the adapted rendering parameters is output (380). In implementations, the adapted visual neuromodulatory codes may be used in a method to deliver visual neuromodulatory codes (see, e.g., FIG. 4 and related description below).

As explained above, the outcome function (i.e., objective function) may be expressed in terms of neurophysiological features calculated from rate and/or blood pressure, e.g., heart rate variability and ratio systolic and diastolic blood pressure, each multiplied by scaling coefficients to produce a “score” to evaluate the rendering parameters in terms of target criteria, e.g., by determining a difference between the outcome function and a target value, threshold, and/or characteristic that is indicative of a desirable state or condition. Thus, the outcome function can be indicative of a therapeutic effectiveness of the visual neuromodulatory code.

As further discussed above (see, e.g., the discussion of FIG. 1), the system 100 provides for the generalization of visual neuromodulatory codes from a wide-ranging population of subjects and/or users. In the generalization process, optimized image parameters are processed to produce a generalized set of image parameters which have a high likelihood of being effective for a large number of users. In some embodiments, the outcome function may be indicative of a degree of generalizability, among the plurality of subjects, of the therapeutic effectiveness of the visual neuromodulatory code. For example, the outcome function may be defined to have a parameter relating to the variance of measure sensor data. This would allow the method to optimize for both therapeutic effect and generalizability.

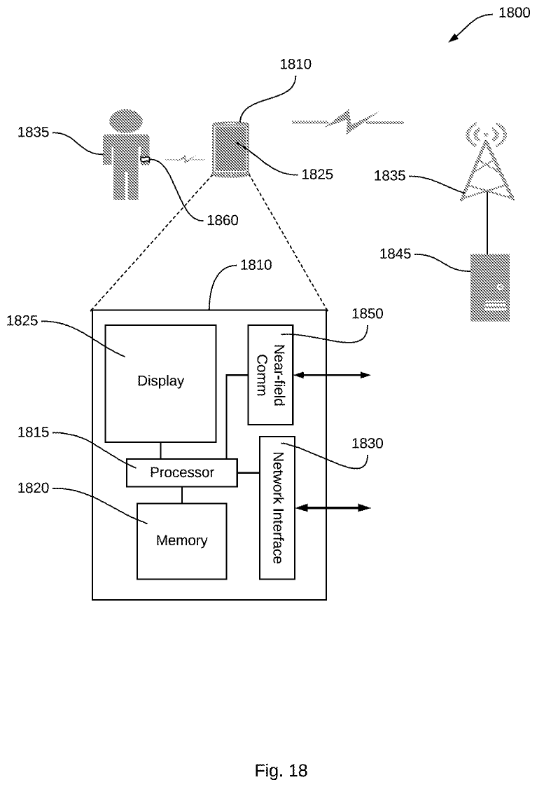

FIG. 4 depicts an embodiment of a method 400, usable with the system of FIG. 18, to provide visual neuromodulatory codes. The method 400 includes retrieving adapted visual neuromodulatory codes, which are adapted to produce physiological responses having therapeutic or performance-enhancing effects (410). The method 400 further includes outputting to an electronic display of a user device the adapted visual neuromodulatory codes (420). In implementations, the one or more adapted visual neuromodulatory codes may be generated, for example, according to the method of FIG. 3, discussed above.

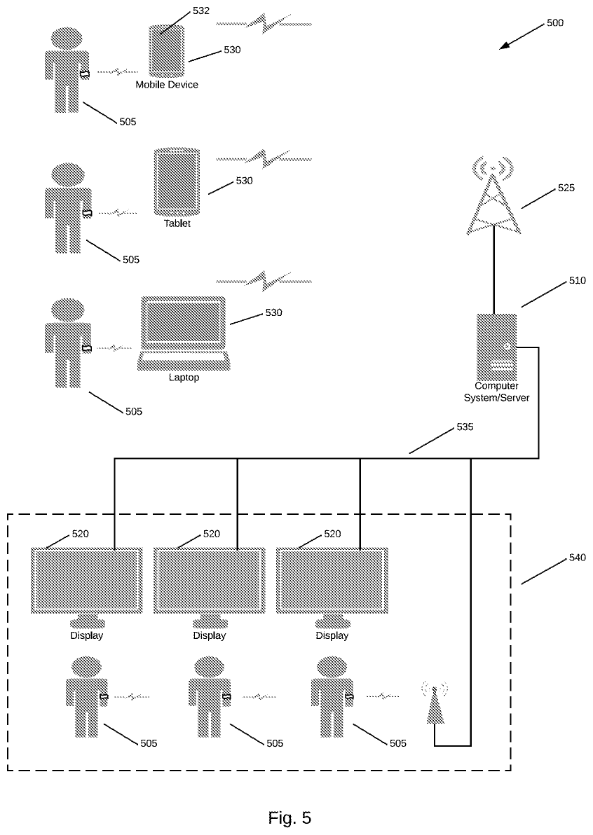

FIG. 5 depicts an embodiment of a system 500 to generate a visual stimulus, using visual codes displayed to a group of participants 505, to produce physiological responses having therapeutic or performance-enhancing effects. The system 500 is processor-based and may include a network-connected computer system/server 510 (and/or other types of computer systems) having at least one processor and memory/storage (e.g., non-transitory processor-readable medium such as random-access memory, read-only memory, and flash memory, as well as magnetic disk and other forms of electronic data storage). The memory/storage stores processor-executable instructions and data which, when executed by the at least one processor, cause the at least one processor to perform the necessary functions for the system to generate and provide to a user the visual stimulus.

A visual code or codes may be generated based on feedback from one or more participants 505 and used as the visual stimulus to produce physiological responses having therapeutic or performance-enhancing effects. The visual stimulus, or stimuli, generated in this manner may, inter alia, effect beneficial changes in specific human emotional, physiological, interoceptive, and/or behavioral states. The visual codes may be implemented in various forms and developed using various techniques, as described in further detail below. In alternative embodiments, other forms of stimuli may be used in conjunction with, or in lieu of, visual neuromodulatory codes, such as audio, sensory, chemical, and physical forms of stimulus

The visual code or codes are displayed to a group of participants 505—either individually or as a group—using electronic displays 520. For example, the server 510 may be connected via a network 525 to a number of personal electronic devices 530, such as mobile phones, tablets, and/or other types of computer systems and devices. The participants 505 may individually view the visual codes on an electronic display 532 of a personal electronic device 530, such as a mobile phone, simultaneously or at different times, i.e., the viewing by one user need not be done at the same time as other users in the group. The personal electronic device may be a wearable device, such as a fitness watch with a display or a pair of glasses that display images, e.g., virtual reality glasses, or other types of augmented-reality interfaces. In some cases, the visual code may be incorporated in content generated by an application running on the personal electronic device 530, such as a web browser. In such a case, the visual code may be overlaid on content displayed by the web browser, e.g., a webpage, so as to be unnoticed by a typical user.

Alternatively, the participants 505 may participate as a group in viewing the visual codes in a group setting on a single display or individual displays for each participant. In such a case, the server may be connected via a network 535 (or 525) to one or more electronic displays which allow for viewing of visual neuromodulatory codes by users in one or more facilities 540 set up for individual and/or group testing.

In some cases, the visual codes may be based at least in part on representational images. In other cases, the visual codes may be formed in a manner that avoids representational imagery. Indeed, the visual codes may incorporate content which is adapted to be perceived subliminally, as opposed to consciously. A “candidate” visual code may be used as an initial or intermediate iteration of the visual code. The candidate visual code, as described in further detail below, may be similar or identical in form and function to the visual code but may be generated by a different system and/or method.

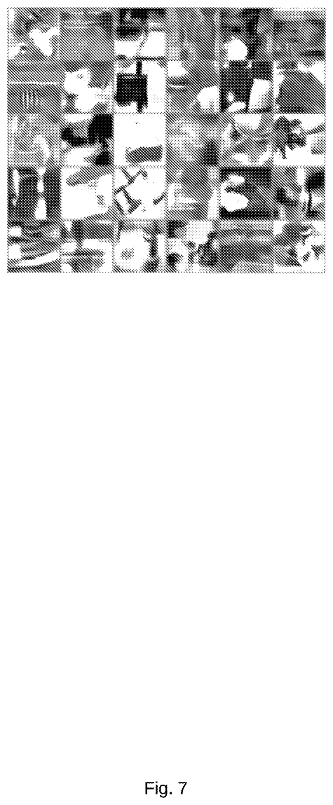

As shown in FIG. 7, the generation of images may start from an initial population of images (e.g., 40 images) created from random achromatic textures constructed from a set of textures which are derived from randomly sampled photographs of natural objects on a gray background. An initial set of “all-zero codes” can be optimized for pixel-wise loss between the synthesized images and the target images using backpropagation through a generative network for a number of iterations, with a linearly decreasing learning rate. The resulting image codes produced are, to an extent, blurred versions of the target images, due to the pixel-wise loss function, thereby producing a set of initial images having quasi-random textures.

Neuronal responses to each synthetic image and/or physiological feedback data indicative of responses of a user, or group of participants, during display of each synthetic image, are used to score the image codes. In each generation, images may be generated from the top (e.g., top 10) image codes from the previous generation, unchanged, plus new image codes (e.g., 30 new image codes) generated by mutation and recombination of all the codes from the preceding generation selected, for example, on the basis of feedback data indicative of responses of a user, or group of participants, during display of the image codes. In disclosed embodiments, images may also be evaluated using an artificial neural network as a model of biological neurons.

In some implementations, the visual codes may be incorporated in a video displayed to the users. In such a case, the visual codes may appear in the video for a sufficiently short duration so that the visual codes are not consciously noticed by the user or users. In various implementations, one or more of the visual codes may encompass all pixels of an image “frame,” i.e., individual image of the set of images of which the video is composed, such that the video is blanked for a sufficiently short duration so that the user does not notice that the video has been blanked. In some cases, the visual code or codes cannot be consciously identified by the user while viewing the video. Pixels forming a visual code may be arranged in groups that are not discernible from pixels of a remainder of an image in the video. For example, pixels of a visual code may be arranged in groups that are sufficiently small so that the visual code cannot be consciously noticed when viewed by a typical user.

The displayed visual code or codes are adapted to produce physiological responses having therapeutic or performance-enhancing effects. For example, the visual code may be the product of iterations of the systems and methods disclosed herein to generate visual codes for particular neural responses or the visual code may be the product of other types of systems and methods. In particular implementations, the neural response may be one that affects one or more of the following: an emotional state, a brain state, a physiological state, an interoceptive state, and a behavioral state. In some cases, displaying the visual code or codes to the group of participants may induce a reaction in at least one user of the group of participants which may, in turn, result in one or more of the following: an emotional change, a physiological change, an interoceptive change, and a behavioral change. Furthermore, the induced reaction may result in one or more of the following: enhanced alertness, reduced anxiety, reduced pain, reduced depression, migraine relief, fear relief, and increased happiness.

As noted above, the visual code or codes may be based at least in part on a candidate visual code which is iteratively generated based on measured brain state and/or brain activity data. For example, the candidate visual code may be generated based at least in part on iterations in which the system receives a first set of brain state data and/or brain activity data measured while a participant is in a target state, e.g., a target emotional state. The first set of brain state data and/or brain activity data forms, in effect, a target for measured brain state/activity. With this point of reference, the candidate visual code is displayed to the participant while the participant is in a current state, i.e., a state other than the target state. The system receives a second set of brain state data and/or brain activity data measured during the displaying of the candidate visual code while the participant is in the current state. Based at least in part on a determined effectiveness of the candidate visual code, as described in further detail below, the system outputs the candidate visual code to be used as the visual stimulus or perturbs the candidate visual code and performs a further iteration.

The user devices also include, or are configured to communicate with, sensors to perform various types of physiological and brain state and activity measurements. This allows the system to receive feedback data indicative of responses of a user, or group of participants, during display of the visual codes to the users. The system performs analysis of the received feedback data indicative of the responses to produce various statistics and parameters, such as parameters indicative of a generalizable effect of the visual codes with respect to the neurological and/or physiological responses having therapeutic effects in users (or group of participants) and—by extension—other users who have not participated in such testing.

In particular implementations, the received feedback data may be obtained from a wearable device, e.g., a fitness band/watch, having sensors to measure physiological characteristics of the group of participants. The received feedback data may include one or more of the following: electrocardiogram (EKG) measurement data, pulse rate data, galvanic skin response, and blood pressure data. Furthermore, human behavioral responses may be obtained using video and/or audio monitoring, such as, for example, blinking, gaze focusing, and posture/gestures. In some cases, the received feedback data includes data characterizing one or more of the following: an emotional state, a brain state, a physiological state, an interoceptive state, and a behavioral state.

In particular implementations, the system may obtain physiological data, and other forms of characterizing data, from a group of participants to determine a respective baseline state of each user. The obtained physiological data may be used by the system to normalize the received feedback data from the group of participants based at least in part on the respective determined baseline state of each user. In some cases, the determined baseline states of the users may be used to, in effect, remediate a state in which the user is not able to provide high quality feedback data, such as, for example, if a user is in a depressed, inattentive, or agitated state. This may be done by providing known stimulus or stimuli to a particular user to induce a modified baseline state in the user. The known stimulus or stimuli may take various forms, such as visual, video, sound, sensory, chemical, and physical forms of stimulus.

Based on the parameters (e.g., parameters indicative of the generalizable effect of the visual codes) and/or statistics resulting from the analysis of the user feedback data for particular visual codes, a selection may be made as to whether to use the particular visual codes as the visual stimulus (e.g., as in the methods to provide a visual stimulus described herein) or to perform further iterations. For example, the selection may be based at least in part on comparing a parameter indicative of the generalizable effect of the visual code to defined criteria. In some cases, the parameter indicative of the generalizable effect of the visual code may be based at least in part on a measure of commonality of the neural responses among the group of participants. For example, the parameter indicative of the generalizable effect of the visual code may represent a percentage of users of the group of participants who meet one or more defined criteria for neural responses.

In the case of performing further iterations, the system may perform various mathematical operations on the visual codes, such as perturbing the visual codes and repeating the displaying of the visual codes, the receiving of the feedback data, and the analyzing of the received feedback data indicative of the responses of the group of participants to produce, inter alia, parameters indicative of the generalizable effect of the visual codes. In particular implementations, the perturbing of the visual codes may be performed using a machine learning model, a neural network, a convolutional neural network, a deep feedforward artificial neural network, an adversarial neural network, and/or an ensemble of neural networks. In some cases, the perturbing of the visual codes may be performed using an adversarial machine learning model which is trained to avoid representational images and/or semantic content to encourage generalizability and avoid cultural or personal bias.

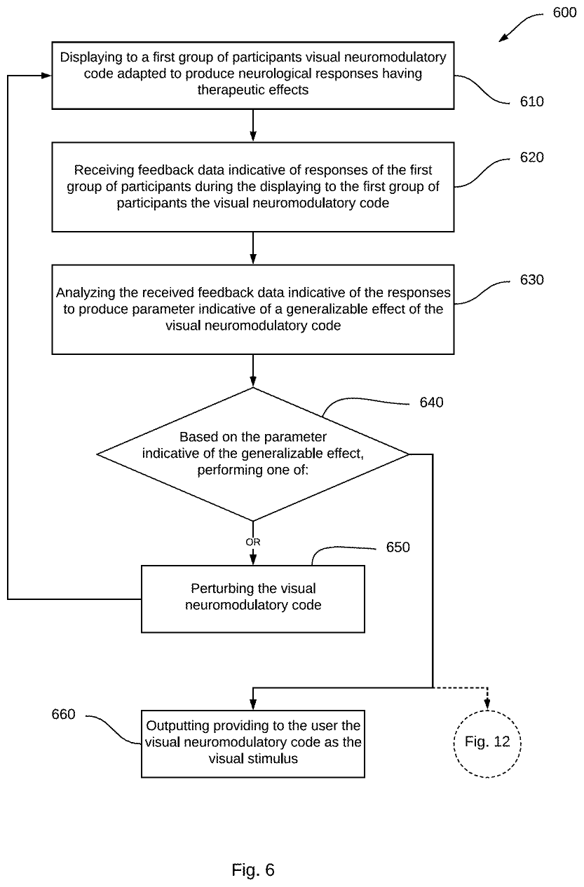

FIG. 6 depicts an embodiment of a method 600 to generate and provide to a user a visual stimulus to produce physiological responses having therapeutic or performance-enhancing effects. The disclosed method 600 is usable in a system such as that shown in FIG. 5, which is described above.

The method 600 includes displaying to a first group of participants (using one or more electronic displays) at least one visual code, at least one visual code adapted to produce physiological responses having therapeutic or performance-enhancing effects (610). The method 600 further includes receiving feedback data indicative of responses of the first group of participants during the displaying to the first group of participants the at least one visual code (620). The method 600 further includes analyzing the received feedback data indicative of the responses to produce at least one parameter indicative of a generalizable effect of the at least one visual code with respect to the neurological responses having therapeutic or performance-enhancing effects in participants of the first group of participants (630).

Based at least in part on the at least one parameter indicative of the generalizable effect of the at least one visual code, the method further includes performing one of: (i) outputting the at least one visual code as the visual stimulus, and (ii) perturbing the at least one visual code and repeating the displaying of the at least one visual code, the receiving the feedback data, and the analyzing the received feedback data indicative of the responses of the first group of participants to produce the at least one parameter indicative of the generalizable effect.

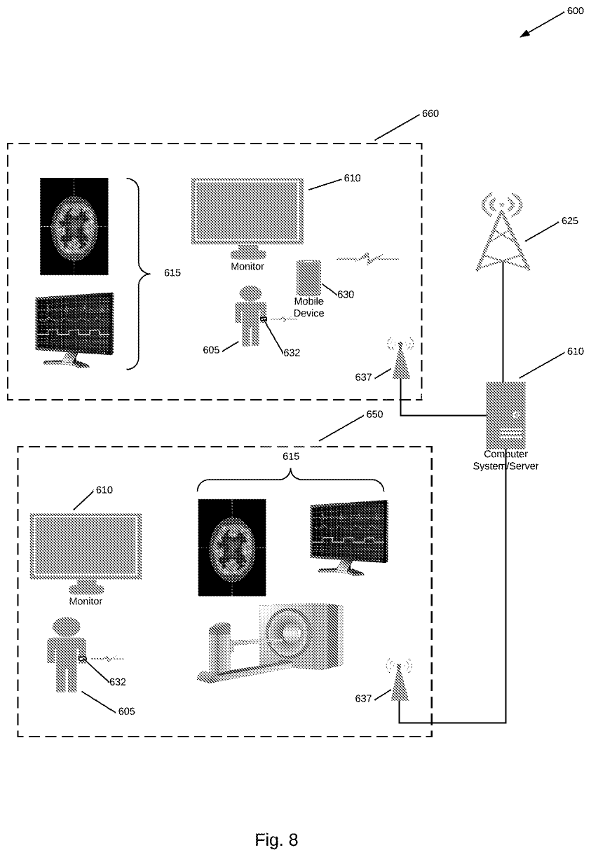

FIG. 8 depicts an embodiment of a system 600 to generate a visual stimulus, using brain state data and/or brain activity data measured while visual codes are displayed to a participant 605 in a target state and a current state, to produce physiological responses having therapeutic or performance-enhancing effects. The system 600 is processor-based and may include a network-connected computer system/server 610, or other type of computer system, having at least one processor and memory/storage. The memory/storage stores processor-executable instructions and data which, when executed by the at least one processor, cause the at least one processor to perform the necessary functions for the system to generate and provide to the user the visual stimulus.

In particular implementations, the computer system/server 610 is connected via a network 625 to a number of personal electronic devices 630, such as mobile phones and tablets, and computer systems. In some cases, the server may be connected via a network to one or more electronic displays which allow for viewing of visual neuromodulatory codes by users in a facility set up for individual and/or group testing, e.g., as discussed above with respect to FIGS. 5 and 6. A visual code may be generated based on feedback from one or more users and used as the visual stimulus to produce physiological responses having therapeutic or performance-enhancing effects, as discussed above.

The system 600 receives a first set of brain state data and/or brain activity data measured, e.g., using a first test set up 650 including a display 610 and various types of brain state and/or brain activity measurement equipment 615, while a test participant 605 is in a target state, e.g., a target emotional state. For example, the target state may be one in which the participant experiences enhanced alertness, reduced anxiety, reduced pain, reduced depression, migraine relief, fear relief, increased happiness, and/or various other positive, desirable states and/or various cognitive functions. The first set of brain state/activity data, thus, serves as a reference against which other measured sets of brain/activity can be compared to assess the effectiveness of a particular visual stimulus in achieving a desired state. The brain state data and/or brain activity data may include, inter alia, data acquired from one or more of the following: electroencephalogram (EEG), quantitative EEG, magnetoencephalography (MEG), single-photon emission computed tomography (SPECT), positron emission tomography (PET), functional magnetic resonance imaging (fMRI), and functional near-infrared spectroscopy (fNIRS)—measured while the participant is present in a facility equipped to make such measurements (e.g., a facility equipped with the first test set up 650). Various other types of physiological and/or neurological measurements may be used. Measurements of this type may be done in conjunction with an induced target state, as the participant will likely be present in the facility for a limited time.

The target state may be induced in the participant 605 by providing known stimulus or stimuli, which may be in the form of visual neuromodulatory codes, as discussed above, and/or various other forms of stimulus, e.g., visual, video, sound, sensory, chemical, and physical, etc. Alternatively, the target state may be achieved in the participant 605 by monitoring naturally occurring states, e.g., emotional states, experienced by the participant over a defined time period (e.g., a day, week, month, etc.) in which the participant is likely to experience a variety of emotional states. In such a case, the system 600 receives data indicative of one or more states (e.g., brain, emotional, cognitive, etc.) of the participant 605 and detects when the participant 605 is in the defined target state.

The system further displays to the participant 605, using an electronic display 610, a candidate visual code while the participant 605 is in a current state, the current state being different than the target state. For example, the participant 605 may be experiencing depression in a current state, as opposed to reduced depression and/or increased happiness in the target state. In particular implementations, the candidate visual code may be based at least in part on or more initial visual codes which are iteratively generated based at least in part on received feedback data indicative of responses of a group of participants during displaying of the one or more initial visual codes to the group of participants, as discussed above with respect to FIGS. 5 and 6.

The system 600 receives a second set of brain state data and/or brain activity data measured, e.g., using a second test set up 660 including a display 610 and various types of brain state and/or brain activity measurement equipment 615, during the display of the candidate visual code to the participant 605. As above, the brain state data and/or brain activity data may include, inter alia, data acquired from one or more of the following: electroencephalogram (EEG), quantitative EEG, magnetoencephalography (MEG), single-photon emission computed tomography (SPECT), positron emission tomography (PET), functional magnetic resonance imaging (fMRI), and functional near-infrared spectroscopy (fNIRS). It should be noted that psychiatric symptoms are produced by the patient's perception and subjective experience. Nevertheless, this does not preclude attempts to identify, describe, and correctly quantify this symptomatology using, for example, psychometric measures, cognitive and neuropsychological tests, symptom rating scales, various laboratory measures, such as, neuroendocrine assays, evoked potentials, sleep studies, brain imaging, etc. The brain imaging may include functional imaging (see examples above) and/or structural imaging, e.g., MRI, etc. In particular implementations, both the first and the second sets of brain state data and/or brain activity data may be obtained using the same test set up, i.e., either the first test set up 650 or the second test set up 660.

The system 600 performs an analysis the first set of brain state/activity data, i.e., the target state data, and the second set of brain state/activity data to produce at least one parameter indicative of an effectiveness of the candidate visual code with respect to the participant 605. For example, the participant 605 may provide feedback, such as survey responses and/or qualitative state indications using a personal electronic device 630, during the target state (i.e., the desired state) and during the current state. In addition, various types of measured feedback data may be obtained (i.e., in addition to the imaging data mentioned above) while the participant 605 is in the target and/or current state, such as electrocardiogram (EKG) measurement data, pulse rate data, blood pressure data, etc. The received feedback data may be obtained from a scale, an electronic questionnaire and a wearable device 632, e.g., a fitness band/watch, having sensors to measure physiological characteristics of the group of participant and communication features to communicate with the system 600, e.g., via a wireless link 637. Analysis of such information can provide parameters and/or statistics indicative of an effectiveness of the candidate visual code with respect to the participant.

Based at least in part on the parameters and/or statistics indicative of the effectiveness of the candidate visual code, the system 600 outputs the candidate visual code as the visual stimulus or performs a further iteration. In the latter case, the candidate visual code is perturbed (i.e., algorithmically modified, adjusted, adapted, randomized, etc.). In particular implementations, the perturbing of the candidate visual code may be performed using a machine learning model, a neural network, a convolutional neural network, a deep feedforward artificial neural network, an adversarial neural network, and/or an ensemble of neural networks. The displaying of the candidate visual code to the participant is repeated and the system receives a further set of brain state/activity data measured during the displaying of the candidate visual code. Analysis is again performed to determine whether to output candidate visual code as the visual stimulus or to perform a further iteration.

In particular implementations, the system may generate a candidate visual code from a set of “base” visual codes. In such a case, the system iteratively generates base visual codes having randomized characteristics, such as texture, color, geometry, etc. Neural responses to the base visual codes are obtained and analyzed. For example, the codes may be displayed to a group of participants with feedback data such as electrocardiogram (EKG) measurement data, pulse rate data, blood pressure data, etc., being obtained. As a further example, the codes may be displayed to participants with feedback data such as electroencephalogram (EEG) data, functional magnetic resonance imaging (fMRI) data, and magnetoencephalography (MEG) data being obtained. Based at least in part on the result of the analysis of the neural responses to the base visual codes, the system outputs a base visual code as the candidate visual code or perturbs one or more of the base visual codes and performs a further iteration. In particular implementations, the perturbing of the base visual codes may be performed using at is performed using at least one of: a machine learning model, a neural network, a convolutional neural network, a deep feedforward artificial neural network, an adversarial neural network, and an ensemble of neural networks.

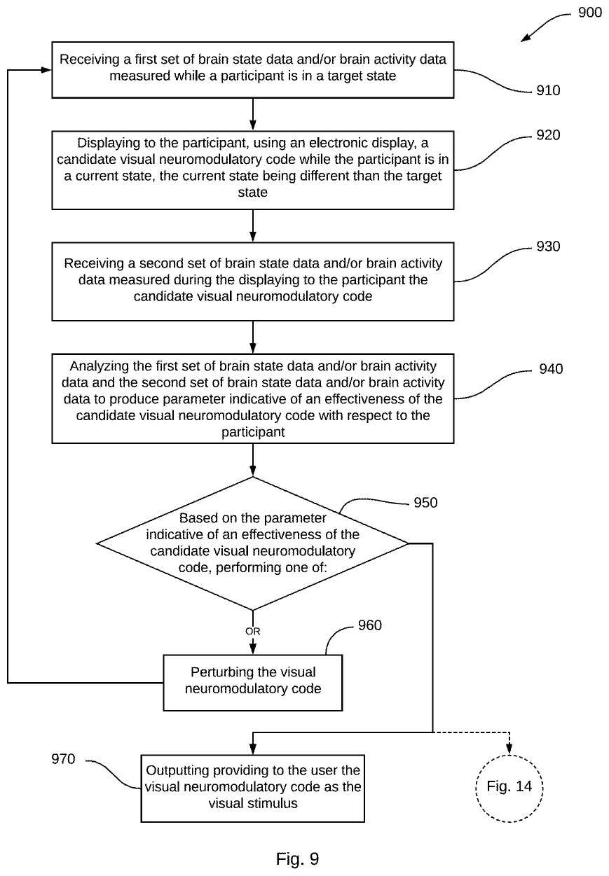

FIG. 9 depicts an embodiment of a method 900 to generate and provide to a user a visual stimulus to produce physiological responses having therapeutic or performance-enhancing effects. The disclosed method is usable in a system such as that shown in FIG. 8, which is described above.

The method 900 includes receiving a first set of brain state data and/or brain activity data measured while a participant is in a target state (910). The method 900 further includes displaying to the participant (using an electronic display) a candidate visual code while the participant is in a current state, the current state being different than the target state (920). The method 900 further includes receiving a second set of brain state data and/or brain activity data measured during the displaying to the participant the candidate visual code (930). The method 900 further includes analyzing the first set of brain state data and/or brain activity data and the second set of brain state data and/or brain activity data to produce at least one parameter indicative of an effectiveness of the candidate visual code with respect to the participant (940).

Based at least in part on the at least one parameter indicative of an effectiveness of the candidate visual code, the method further includes performing (950) one of: (i) outputting the candidate visual code as the visual stimulus (970), and (ii) perturbing the candidate visual code and repeating the displaying to the participant the candidate visual code, the receiving the second set of brain state data and/or brain activity data measured during the displaying to the participant the candidate visual code, and the analyzing the first set of brain state data and/or brain activity data and the second set of brain state data and/or brain activity data (960).

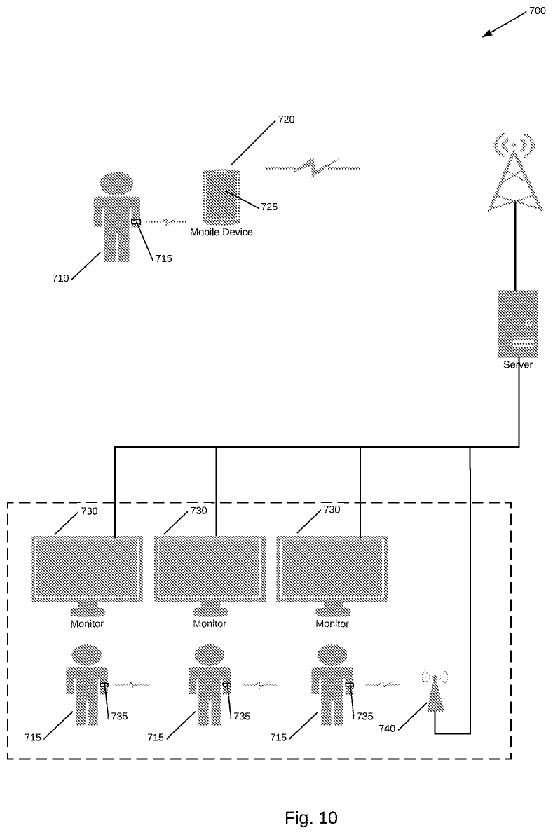

FIG. 10 depicts an embodiment of a 700 system to deliver a visual stimulus to a user 710, generated using visual codes displayed to a group of participants 715, to produce physiological and/or neurological responses. The system 700 is processor-based and may include a network-connected personal electronic device, e.g., a mobile device 720, or other type of network-connected user device (e.g., tablet, desktop computer, etc.), having and electronic display and at least one processor and memory/storage. The memory/storage stores processor-executable instructions and data which, when executed by the at least one processor, cause the at least one processor to perform the necessary functions for the system to provide the visual stimulus.

The system 700 outputs a visual code or codes to the electronic display 725 of the personal electronic device, e.g., mobile device 720. The visual codes are adapted to act as the visual stimulus to produce physiological responses having therapeutic or performance-enhancing effects. In particular implementations, the neural response may be one that affects an emotional state, a brain state, a physiological state, an interoceptive state, and/or a behavioral state of the user. The outputting to the electronic display 725, e.g., to the electronic display of the user's mobile device 720 (or other type of personal electronic device), the visual code or codes induces a reaction in the user resulting, for example, in an emotional change, a physiological change, an interoceptive change, and/or a behavioral change. The change in state and/or induced reaction in the user 710 may result in, inter alia, enhanced alertness, reduced anxiety, reduced pain, reduced depression, migraine relief, fear relief, and increased happiness. In implementations, the therapeutic effect may be usable as a substitute for, or adjunct to, anesthesia.

There are various methods of delivery for the visual neuromodulatory codes, including running in the background, “focused delivery” (e.g., user focuses on stimulus for a determined time with full attention), and overlaid—additive (e.g., a largely translucent layer overlaid on video or web browser content). In such an implementation, the visual code overlaid on the displayable content may make a screen of the electronic device appear to be noisier, but a user generally would not notice the content of a visual code presented in this manner.

The visual codes are generated by iteratively performing a method such as the method described above with respect to FIGS. 5 and 6. In such a case, the method includes displaying to a group of participants 715 at least one test visual code, the at least one test visual code being adapted to activate the neural response to produce physiological responses having therapeutic or performance-enhancing effects.

The method further includes receiving feedback data indicative of responses of the group of participants 715 during the simultaneous displaying (e.g., using one or more electronic displays 730) to the group of participants 715 the at least one test visual code. The received feedback data may be obtained from a biomedical sensor, such as a wearable device 735 (e.g., smart glasses, watches, fitness bands/watches, wristbands, running shoes, rings, armbands, belts, helmets, buttons, etc.) having sensors to measure physiological characteristics of the participants 715 and communication features to communicate with the system 700, e.g., via a wireless link 740.

In general, biomedical sensors are electronic devices that transduce biomedical signals indicative of human physiology, e.g., brain waves and heat beats, into measurable electrical signals. Biomedical sensors can be divided into three categories depending on the type of human physiological information to be detected: physical, chemical, and biological. Physical sensors quantify physical phenomena such as motion, force, pressure, temperature, and electric voltages and currents—they are used to measure and monitor physiologic properties such as physical blood pressure, respiration, pulse, body temperature, heart sound, respiratory rate, blood viscosity, flow rate, flow rate, etc. Chemical sensors are utilized to measure chemical parameters such as oxygen and carbon dioxide concentration in the human metabolism, pH value, and ion levels in bodily fluids (e.g., Na⁺, K⁺, Ca²⁺, and Cl⁻). Biological sensors (i.e., “biosensors”) are used to detect biological parameters, such as tissues, cells, enzymes, antigens, antibodies, receptors, hormones, cholic acid, acetylcholine, serotonin, DNA and RNA, and other proteins and biomarkers.

The method further includes analyzing the received feedback data indicative of the responses to produce at least one parameter indicative of a generalizable effect of the at least one visual code with respect to the neurological responses having therapeutic effects in participants of the first group of participants. Based at least in part on the at least one parameter indicative of the generalizable effect of the at least one visual code, the method further includes performing one of: (i) outputting the at least one test visual code as the at least one visual code, and (ii) perturbing the at least one test visual code and performing a further iteration.

Referring again to FIG. 10, the system 700 obtains user feedback data indicative of responses of the user 710 during the outputting of the visual codes to the electronic display 725 of the mobile device 720. In particular implementations, the user feedback data may be obtained from sensors and/or user input. For example, the mobile device 720 may be wirelessly connected to a wearable device 740, e.g., a fitness band or watch, having sensors which measure physiological conditions of the user 710. The obtained user feedback data may include data characterizing an emotional state, a brain state, a physiological state, an interoceptive state, and/or a behavioral state of the user. Furthermore, the obtained user feedback data may include electrocardiogram (EKG) measurement data, pulse rate data, blood pressure data, etc.

In particular implementations, the system 700 may analyze the obtained user feedback data indicative of the responses of the user 710 to produce one or more parameters indicative of an effectiveness of the visual code or codes. In such a case, the system would iteratively perform (based at least in part on the at least one parameter indicative of the effectiveness of the at least one visual code) one of: (i) maintaining the visual code or codes as the visual stimulus, and (ii) perturbing the visual code or codes and performing a further iteration.

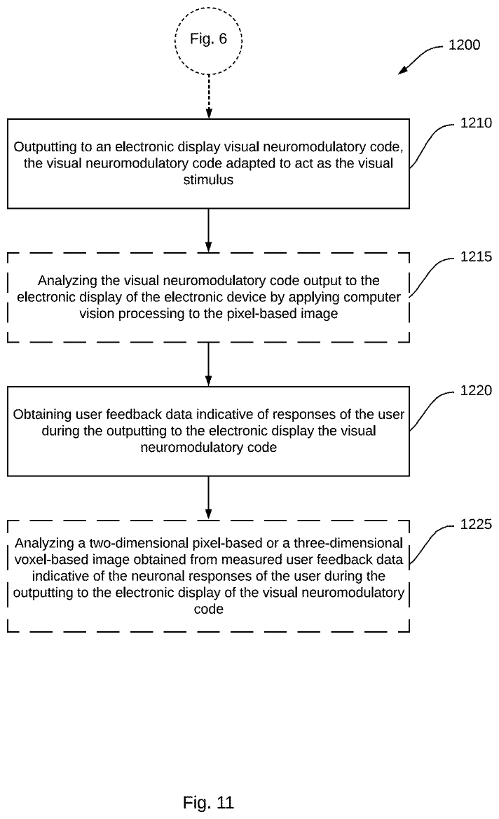

FIG. 11 depicts an embodiment of a method 1200 to deliver (i.e., provide) a visual stimulus to produce physiological responses and useful in creating sensory brain maps, biotyping, and diagnostics. The disclosed method is usable in a system such as that shown in FIG. 10, which is described above. The method 1200 includes outputting to an electronic display of an electronic device at least one visual code, which, for example, may be in the form of a sequence of video frames. The at least one visual code is adapted to act as the visual stimulus to produce physiological/neurological responses (1210). The method further includes obtaining user feedback data indicative of responses of the user during the outputting to the electronic display the at least one visual code (1220). In implementations, the at least one visual code may be generated using, for example, the method to generate a visual stimulus of FIG. 6, discussed above.

Disclosed embodiments may include an end-to-end computer vision platform in which visual stimuli, e.g., visual neuromodulatory codes, are created by computational graphics and then characterized, i.e., parameterized, using computer vision techniques. Computer vision techniques typically involve a type of machine learning called “deep learning” and a convolutional neural network (CNN), which, in effect, breaks down images down into pixels that are given tags or labels. It uses the labels to perform convolutions and makes predictions. The neural network runs convolutions and checks the accuracy of the predictions in a series of iterations. Much like a human making out an image at a distance, a CNN first discerns hard edges and simple shapes, then fills in information as it runs iterations of its predictions. A CNN may be used to understand single images, or sequences of images (e.g., a video sequence), such as a visual neuromodulatory code or “dynamic” neuromodulatory code. A recurrent neural network (RNN) may be used in a similar way for a series/sequence of images.

In embodiments using computer vision techniques, the method 1200 may further include analyzing the at least one visual neuromodulatory code output to the electronic display of the electronic device by applying computer vision processing to the pixel-based image (1215). The method further may further include analyzing a two-dimensional pixel-based or a three-dimensional voxel-based image obtained from measured user feedback data indicative of the neuronal responses of the user during the outputting to the electronic display of the at least one visual neuromodulatory code (1225).

Using such an approach, the complexity of the graphics being created can be described in highly measurable manner, which allows for description of, for example, movement, shape formation, the complexity of a number of items occurring on a display screen at any one time (e.g., arrangements of items). These aspects, inter alia, can be described by computer vision by creating sophisticated computer vision descriptors. Moreover, in this manner, the graphics (e.g., visual neuromodulatory codes) are parameterized, which allows for control of the creation and presentation of the computational graphics and, thus, the input of the end-to-end computer vision platform.

Underlying the end-to-end computer vision approach, is the notion that the brain may be described as an “optical engine” having a non-unique communication protocol which is geometric in nature—a geometric language to control neuronal populations and neuronal activity—and which is, in a sense, akin to a genetic coding system. In disclosed embodiments, a computer vision system may be used to measure the manner in which this geometric protocol is expressed in the brain, e.g., in terms of neuronal response based on neuro-imaging techniques described herein, thereby providing a basis for examination with greater temporal and positional resolution. In this way, the visual neuromodulatory codes generated on the input side can be characterized using computer vision techniques and analyzed in conjunction with computer vision—based descriptions of neuronal responses in the brain, including the geometric properties and time-based geometry and movement of such measured responses, thereby providing a mapping between the inputs and outputs of the end-to-end computer vision platform. The output, e.g., the neuronal response in the brain, can be measured as described herein using one or more of the following, e.g., quantitative EEG, magnetoencephalography (MEG), functional magnetic resonance imaging (fMRI), and functional near-infrared spectroscopy (fNIRS). Thus, the end-to-end computer vision approach provides for time-based analysis of the visual neuromodulatory codes and the resulting neural responses using the same descriptive mechanisms on both the input and output ends.

In embodiments, in addition to—or in lieu of—using machine learning to understand if there are any patterns of behavior, etc., and Bayesian approaches to analyze pattern matching, the computer vision approach may be used to analyze the geometry, especially repeating and/or fast changing geometry, which may be difficult to analyze using other techniques. Using the computer vision techniques allows for a form of geometric classification, which is important because an output, e.g., a neuronal response, of a particular geometry may be more significant than one having the greatest amplitude. For example, studies have shown that perception, e.g., pain perception, is represented in many distinct locations of the brain thereby forming a matrix-like geometry—the arrangement of which may vary with time. The geometry of the output may be analogized to a dynamic geometric form akin to a crystal. Such geometric classifications can be analyzed over time to find patterns in a much faster and precise way. This approach may be more efficient compared to analysis using machine learning which, in most cases, looks for relationships between inputs and outputs in their entirety, i.e., as a mass of pixels and/or voxels, and would not necessarily consider distinctive geometric patterns to be of greater significance than portions inputs and outputs having the greatest amplitude. Thus, analysis of outputs (e.g., measured neuronal responses) may be performed to develop classification systems for geometries in a manner akin to the classification systems for the visual stimuli, e.g., visual neuromodulatory codes) described herein. This approach allows for the determination of transformations between inputs and outputs to allow an efficient model to be created for the end-to-end system.

Using computer vision techniques in the analysis of measured neuronal responses, as described above, is advantageous in that it takes into account the timing and the relationship between the locations where individual elements, i.e., areas and/or volumes, of the neuronal response are taking place in the brain and provides for fast and efficient description of the fast-changing temporal geometry of optical energy. Measurement techniques such as magnetic resonance imaging (MRI), on the other hand, merely provide information on position and amplitude of the multitude of individual elements of the neuronal response.

In embodiments, computer vision techniques are applied to measurements made of the neuronal responses of a subject while viewing visual neuromodulatory codes, using techniques described herein, and the computer vision techniques are also applied to the visual neuromodulatory code itself, thereby producing a set of input computer vision parameters and a set of output computer vision parameters. The sets of parameters may be processed, e.g., using machine learning algorithms, as described herein, along with sets of other types of measured data, such as physiological and/or behavioral responses of the subject. This allows for an iterative approach to optimizing the visual neuromodulatory codes to achieve target neuronal, physiological and/or behavioral responses.

In embodiments, measurements of physiological response can be made with respect targets for the physiological readings, such as heart rate or reduce blood pressure, etc. In this manner, an algorithmic process is created involving: (i) input parameters produced using computer vision techniques from visual neuromodulatory codes displayed to a subject; (ii) output parameters produced using computer vision techniques from neuronal response measurements; and (iii) physiological measurements versus physiological targets. This algorithmic process can be used to iteratively refine the rendering parameters used to produce the visual neuromodulatory codes displayed to the subject. Thus, in embodiments, the system can use computer vision descriptors for the visual stimuli and computer vision descriptors for the representation of the neuronal imaging information—linking the two to optimize the system—instead of relying directly on the underlying rendering parameters and physiological measurements.

The application of computer vision techniques to measurements made of the neuronal responses of a subject while viewing visual neuromodulatory codes may include measurements made during induced target states. For example, a target brain state can be induced in a subject by administering pharmacological agents, administering anesthesia, inducing pain or other stimulation, etc., thereby allowing description of an induced state in terms of computer vision geometry. Such geometries can be maintained in a library of measured geometries for use in further analysis.

In embodiments, faster and more accurate imaging technologies will allow smaller elements of visual coding to be deduced and will allow these to be linked to smaller geometric effects in the brain, thereby deriving a finer resolution representation of the geometric communication protocol. In a mapping process, geometric properties of neuronal responses created by blunt inputs can be used to further refine the visual stimuli to produce more fine-grained responses.

In embodiments, systems and methods described herein provide for stimulus-mediated brain mapping from which interpretable brain performance metrics can be derived. Based on such metrics, it is possible to infer changes in brain health due to disorders or to therapeutic actions and to infer the likelihood of effectiveness of drug candidates or other therapeutic actions.

Sensor mapping may be used to characterize brain health and detect changes in brain health due to disorders or due to therapeutic applications to address disorders. In general, a sensor map may be highly dimensional and difficult to directly interpret. However, a sensor map can serve as the foundation of a number of low-dimensional and, hence, more interpretable “inference metrics” derived from it. In embodiments, inference metrics may be derived, each from a unique nonlinear mapping (e.g., a deep neural network) from the stimulus map, Such inference metrics are intended to preserve information required to characterize and detect changes in brain health. Furthermore, inference metrics are useful for discerning root causes of changes in brain health, which can increase the likelihood that particular therapies, including drugs, will be successful. Even if individual inference metrics do not by themselves describe interpretable properties (e.g., bandwidth), the inference metrics provide a low dimensional summary of the sensor map, where the effect of different disorders and the effect of different classes of drugs or other therapies may be represented by different characteristic degrees of variation, including, in some cases, sparseness. Inference metrics may be used together with a multidimensional scoring system to perform biotyping by assigning a score to each combination of disease and therapeutic or drug attribute. For diagnostic applications, a probability score can be assigned for each of a number of disorders, based on a trained mapping between the stimulus map, for example using a Graph Neural Network.

As discussed above, sensory mapping, i.e., stimulus mapping, of the brain may be performed with complex spatiotemporal sensory inputs, which may be composed of a series of “codes,” e.g., visual neuromodulatory codes or spatiotemporal sensory codes. The spatiotemporal sensory inputs, in any given time span, may be a mixture of one or more spatiotemporal sensory codes. In embodiments, the spatiotemporal sensory codes may have a fixed length of a timespan T, and that the totality of the input is created by stringing codes together one after another, potentially with some overlap (e.g., ¼ overlap) in time such that one code crossfades to the next code in the overlap region, e.g., as described by a window function such as a Hamming window. The window function may be thought of as a mixing function meant to reinforce continuity across cross-faded windows, which may be referred to as “overlap add.” Such windows need not be overlapping and, in embodiments, the window length may be one frame. In such a case, the system adjusts on a frame-by-frame basis.

A code can be described both by “encoding data” (or “parameters”) from which a code may be generated, given a particular generation model or algorithm. A code may also be described by a post hoc characterization after its creation by describing, e.g., spatiotemporal landmarks or the shape of the amplitude and phase of the Fourier spectrum. Furthermore, a post hoc characterization may be based on computer vision (e.g., deep neural network) descriptors, as discussed above. In both cases, codes may be considered unique and the similarity between two codes can be determined. In general, given the same generation algorithm or model, identicality or similarity in encoding data implies identicality or similarity of the resulting codes. Furthermore, two codes that are the same or very similar will have the same or very similar post-hoc analysis features, independent of the generation algorithm.

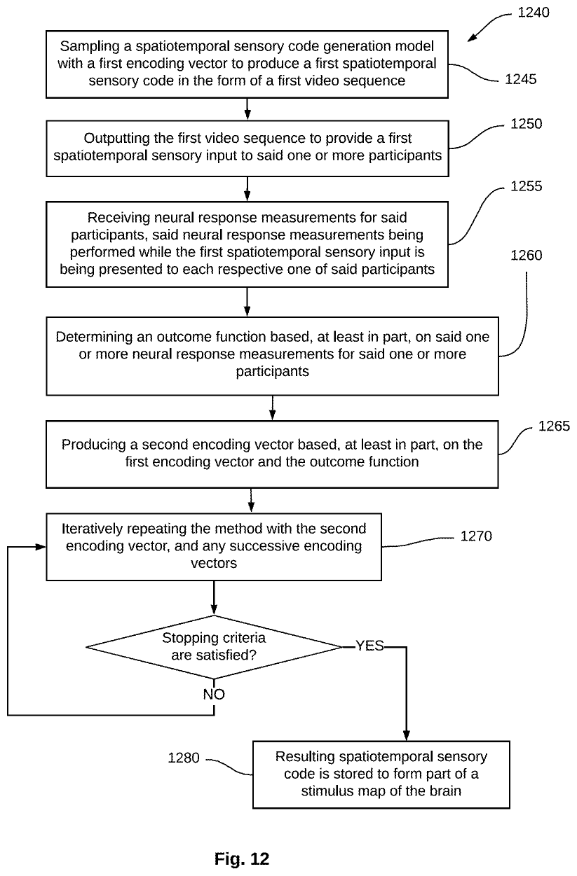

FIG. 12 depicts a method 1240 for providing spatiotemporal sensory inputs to one or more participants to produce a stimulus map of the brain. The method 1240 includes sampling a spatiotemporal sensory code generation model with a first encoding vector to produce a first spatiotemporal sensory code in the form of a first video sequence (1245).

The spatiotemporal sensory codes may be in the form of visual sensory inputs, auditory sensory inputs, and somatosensory inputs. Disclosed embodiments focus on visual inputs, but the methods are also applicable to auditory or somatosensory inputs, as well as any other sensory inputs having a complex temporal or spatiotemporal character.

The generation model may include procedural graphics using input parameters (which may be in the form of an encoding vector comprising an array of input parameters), such as spatial frequencies, temporal frequencies, spatial locations, spatial extents, and translation-based motion vectors. In such a case, a code may be described by superimposed 3-D sinusoidal components modulated by a spatiotemporal envelope. The spatiotemporal sensory code generation model may include a generative adversarial network or deep diffusion model and the first encoding vector points to a location in a latent generation space. Examples of generation models include deep generation models such as Generative Adversarial Networks (GANs) or Deep Diffusion Models. In this case a code is described by an encoding vector that points to a location in a latent generation space. The generation models may be trained to have specific characteristics. For example, the generative models may be adapted to generate non-figurative video having high-order statistics that resemble that of natural scenes. This will result in generated videos that map more closely with the natural statistics of activity sequences in the brain.

The method further includes outputting the first video sequence to provide a first spatiotemporal sensory input to the participants (1250). In embodiments, the spatiotemporal sensory codes, in the form of video sequences, may have a defined time length and partially overlap in time. The first video sequence may have N frames starting from time Tᵢ, in which case the method further includes applying a per-frame window function to the first video sequence and adding the result to an output frame buffer, filling frames from Tᵢ to Tᵢ+N.

The method further includes receiving neural response measurements for the participants, with the neural response measurements being performed in time steps while the first spatiotemporal sensory input is being presented to each respective one of the participants (1255). In embodiments, the neural response measurements may be performed using one or more of the following: electroencephalogram (EEG), quantitative EEG, magnetoencephalography (MEG), single-photon emission computed tomography (SPECT), positron emission tomography (PET), functional magnetic resonance imaging (fMRI), and functional near-infrared spectroscopy (fNIRS). In embodiments, the neural response measurements may be performed using one or more of the following: electrocardiogram (EKG) measurement data, pulse rate data, galvanic skin response, and blood pressure data. An outcome function is determined based, at least in part, on the neural response measurements for the participants (1260).

A second encoding vector is produced based, at least in part, on the first encoding vector and the outcome function (1265). In embodiments, aside from the influence of the measured neural response, the second encoding vector may be produced based on momentum derived from past timesteps and/or may rely on other forms of dynamics (and other mathematical relationships, more generally) to explore the space of possibilities efficiently. The measured neural response, which is characterized to generate the second encoding vector (and successive encoding vectors) to produce video sequences for the output frame buffer, may be given a variable degree of influence over the future trajectory of the encoding vectors. Depending on the nature of the neural response, for example if it appears to be very noisy, the momentum from the past encoding vectors may be given more influence on the future encoding vectors.

The method is iteratively repeated (i.e., said sampling 1245, said outputting 1250, said receiving 1255, and said determining 1260) with the second encoding vector, and any successive encoding vectors until a defined set of stopping criteria for the outcome function is satisfied. (1270). Upon satisfying the defined set of stopping criteria for the outcome function, a resulting spatiotemporal sensory code is stored to form part of the stimulus map of the brain (1280).

Referring again to the sampling of the spatiotemporal sensory code generation model with the first encoding vector (1245), by modifying the “generation data” (e.g., an encoding vector and/or other forms of input parameters) a sequence of codes can be generated that have spatiotemporal variation. As discussed below, in producing a brain map, a sequence of codes (or other input parameters) can be associated with specific physical locations in the brain or may be associated with more complex characterizations of the brain as a whole. The brain mapping methods described herein may be repeated until a majority or all addresses are filled with visual codes, thereby creating a stimulus map of the brain,

In some approaches, brain mapping may involve presenting a series of stimulus inputs, where a small number of parameters vary in the series in a predefined way. A brain map can be produced by characterizing, e.g., one aspect of the neural response, such as firing rate, or firing precision, at a particular location in the brain, as a function of the parameter values. The characteristics of a particular location may be summarized as the parameter values that maximize the neural response, for example. This can be repeated for an array of different locations in the brain.

In other approaches, code sequences may be associated with neural states at particular locations, but more generally may be associated by whole-brain neural states as described by a graph, such as a functional connectome. On a brain graph, more complex neural objective functions may be defined, such as a multivariate cross-coherence (across spectral bands), where a code sequence is associated with maximizing the cross-coherence across one or more pairs of nodes, and different code sequences in the map are associated with cross-coherence patterns that are independent from those of other code sequences in the map. In disclosed embodiments, the generation of effective code sequences is directed by a control algorithm which either steers the parameters in a procedural graphics algorithm, or steers the encoding vector in deep generative models to converge on an effective code. Such control algorithms may be, for example, non-convex control algorithms, including deep reinforcement learning algorithms. In this way a map can be formed by associating each resulting code (i.e., each vector in encoding data space) with its corresponding multivariate graph (i.e., a vector in neural state space), until a neural state space is sufficiently covered. For example, a neural state space defined by a multivariate graph may be partitioned into a N-dimensional grid, where each location is associated with a code resulting from the algorithm.

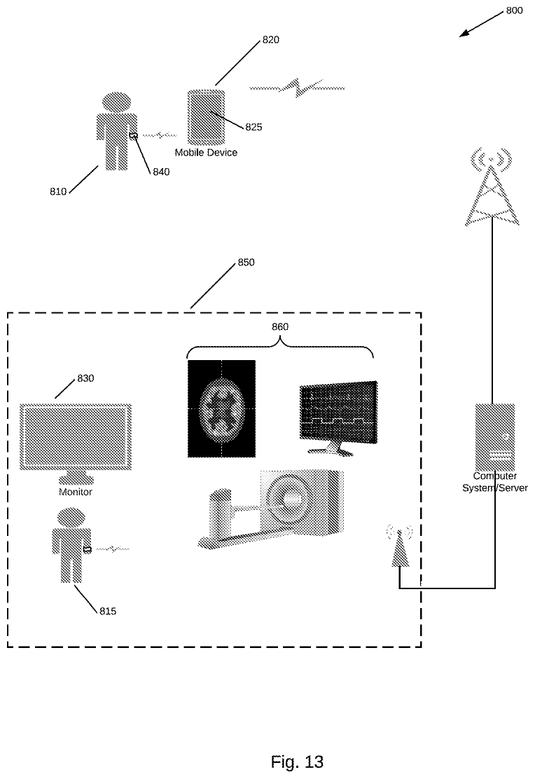

FIG. 13 depicts an embodiment of a system 800 to deliver a visual stimulus to a user 810, generated using brain state data and/or brain activity data measured while visual codes are displayed to a participant in a target state and a current state, to produce physiological responses having therapeutic or performance-enhancing effects. The system 800 is processor-based and may include a network-connected personal electronic device, e.g., a mobile device 820, or other type of network-connected user device (e.g., tablet, desktop computer, etc.), having and electronic display and at least one processor and memory/storage. The memory/storage stores processor-executable instructions and data which, when executed by the at least one processor, cause the at least one processor to perform the necessary functions for the system to provide the visual stimulus.

The system 800 outputs a visual code or codes to the electronic display 825 of the personal electronic device, e.g., mobile device 820. The visual codes are adapted to act as the visual stimulus to produce physiological responses having therapeutic or performance-enhancing effects. In particular implementations, the neural response may be one that affects an emotional state, a brain state, a physiological state, an interoceptive state, and/or a behavioral state of the user. The outputting to the electronic display 825, e.g., to the electronic display of the user's mobile device 820 (or other type of personal electronic device), the visual code or codes induces a reaction in the user resulting, for example, in an emotional change, a physiological change, an interoceptive change, and/or a behavioral change. The change in state and/or induced reaction in the user 810 may result in, inter alia, enhanced alertness, reduced anxiety, reduced pain, reduced depression, migraine relief, fear relief, and increased happiness.

The visual codes are generated by iteratively performing a method such as the method described above with respect to FIGS. 8 and 9. In such a case, the method includes receiving a first set of brain state data and/or brain activity data measured, e.g., using a test set up 850 including a display 830 and various types of brain state and/or brain activity measurement equipment 860, while a participant 815 is in a target state. The method further includes displaying to the participant 815 a candidate visual code (e.g., using one or more electronic displays 830) while the participant 815 is in a current state, the current state being different than the target state. The method further includes receiving a second set of brain state data and/or brain activity data measured, e.g., using the depicted test set up 850 (or a similar test set up), during the displaying to the participant 815 of the candidate visual code. The first set of brain state data and/or brain activity data and the second set of brain state data and/or brain activity data are analyzed to produce at least one parameter indicative of an effectiveness of the candidate visual code with respect to the participant. Based at least in part on the at least one parameter indicative of an effectiveness of the candidate visual code, the method further includes performing one of: (i) outputting the candidate visual code as the visual code, and (ii) perturbing the candidate visual code and performing a further iteration.

The system 800 obtains user feedback data indicative of responses of the user 810 during the outputting of the visual code or codes to the electronic display 825 of the user's mobile device 820. In particular implementations, the user feedback data may be obtained from sensors and/or user input. For example, the mobile device 820 may be wirelessly connected to a wearable device 840, e.g., a fitness band or watch, having sensors which measure physiological conditions of the user 810. The obtained user feedback data may include, inter alia, data characterizing an emotional state, a brain state, a physiological state, an interoceptive state, and a behavioral state. The obtained user feedback data may include, inter alia, electrocardiogram (EKG) measurement data, pulse rate data, and blood pressure data.

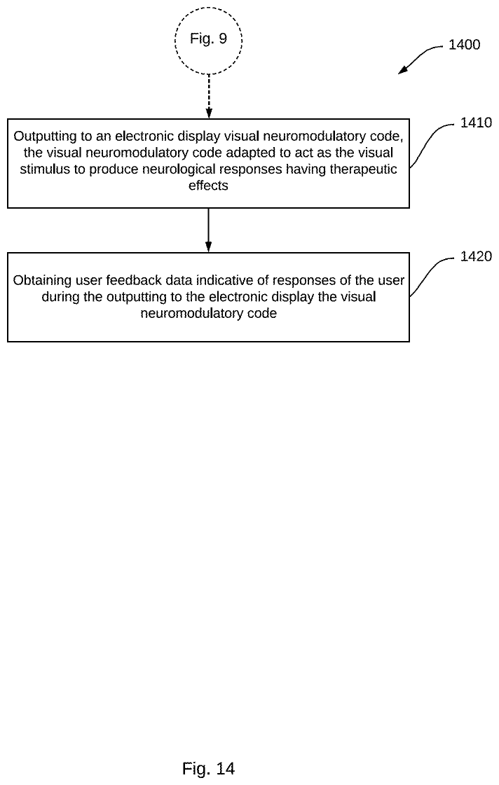

FIG. 14 depicts an embodiment of a method 1400 to deliver (i.e., provide) a visual stimulus to produce physiological responses having therapeutic or performance-enhancing effects. The disclosed method 1400 is usable in a system such as that shown in FIG. 13, which is described above. The method 1400 includes outputting to an electronic display at least one visual code, the at least one visual code adapted to act as the visual stimulus to produce physiological responses having therapeutic or performance-enhancing effects (1410). The method 1400 further includes obtaining user feedback data indicative of responses of the user during the outputting to the electronic display the at least one visual code (1420). In implementations, the at least one visual code may be generated using, for example, the method to generate a visual stimulus to produce physiological responses having therapeutic or performance-enhancing effects of FIG. 9, discussed above.

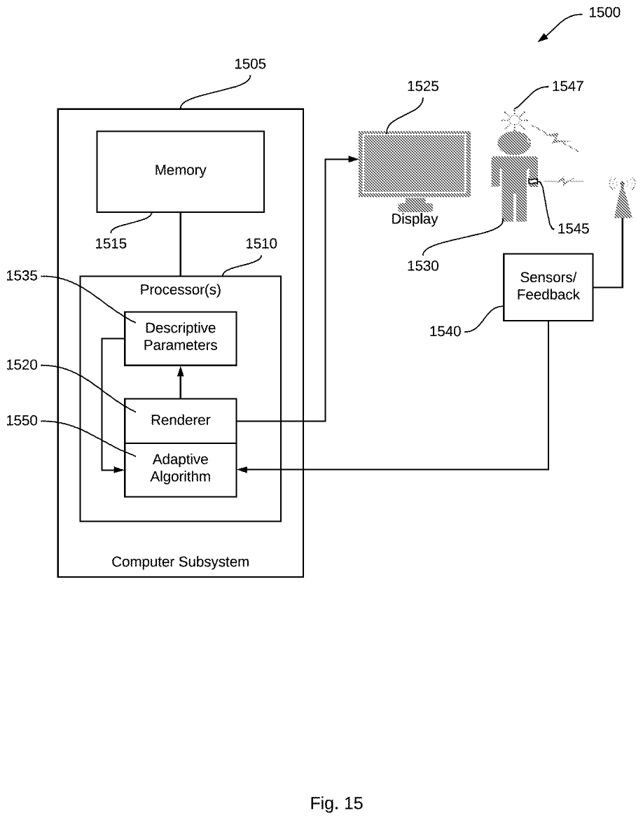

FIG. 15 depicts an embodiment of a system 1500 to generate visual neuromodulatory codes with a closed-loop approach using an optimized descriptive space to produce physiological responses having therapeutic or performance-enhancing effects. The system 1500 includes a computer subsystem 1505 comprising at least one processor 1510 and memory 1515 (e.g., non-transitory processor-readable medium). The memory 1515 stores processor-executable instructions which, when executed by the at least one processor 1510, cause the at least one processor 1510 to perform a method to generate the visual neuromodulatory codes. Specific aspects of the method performed by the processor 1510 are depicted as elements (e.g., code, software modules, and/or processes) within the processor for purposes of discussion only.

The renderer 1520 performs a rendering process to produce images (e.g., sequences of images) to be displayed on the display 1525 by generating video data based on specific inputs. In implementations, the output of the rendering process is a digital image stored as an array of pixels. Each pixel value may be a single scalar component or a vector containing a separate scalar value for each color component. The renderer 1520 may produce (i.e., synthesize) one or more visual neuromodulatory codes (e.g., a sequence of visual neuromodulatory codes) based on an initial set of rendering parameters (i.e., synthesis parameters) stored in the memory 1515. The video data and/or signal resulting from the rendering is output by the computer subsystem 1505 to the display 1525.

The system 1500 is configured to present the visual neuromodulatory codes to at least one subject 1530 by arranging the display 1525 so that it can be viewed by the subject 1530. For example, a video monitor may be provided in a location where it can be accessed by the subject 1530, e.g., a location where other components of the system are located. Alternatively, the video data may be transmitted via a network to be displayed on a video monitor or mobile device of the subject (not shown). In implementations, the subject may be one of the users of the system. In implementations, the visual neuromodulatory codes may be presented to a plurality of subjects, as described with respect to FIGS. 1-4.

In implementations, the system 1500 may present on the display 1525 a dynamic visual neuromodulatory code based on visual neuromodulatory codes. For example, a dynamic visual neuromodulatory code may be formed by combining a number of visual neuromodulatory codes to form a sequence of visual neuromodulatory codes. In a further example, a dynamic visual neuromodulatory code may be adapted to produce at least one of the following effects: a pulsating effect, a zooming effect, a flickering effect, and a color-shift effect. In some cases, the formation of the dynamic visual neuromodulatory code may include processing a set, e.g., a sequence, of visual neuromodulatory codes to produce intermediate images in the sequence of visual neuromodulatory codes. Various techniques, such as interpolation of pixels and gaussian averaging, may be used to produce the intermediate images.

In addition to outputting the visual neuromodulatory codes to the display 1525, the computer subsystem 1505 also includes a descriptive parameters calculator 1535 (e.g., code, a module, and/or a process) which computes values for descriptive parameters in a defined set of descriptive parameters characterizing the visual neuromodulatory codes produced by the renderer. In implementations, the defined set of descriptive parameters used to characterize the visual neuromodulatory codes is selected from a number of candidate sets of descriptive parameters by: rendering visual neuromodulatory codes; computing values of the descriptive parameters of each of the candidate sets of descriptive parameters; and modeling the performance of each of the candidate sets of descriptive parameters. Based on the modeled performance, one of the candidate sets of descriptive parameters is selected and used in the closed-loop process.

In some cases, the selected set of descriptive parameters comprises low-level statistics of visual neuromodulatory codes, including color, motion, brightness, and/or contrast. Another set of descriptive parameters may comprise metrics characterizing visual content of the visual neuromodulatory codes, including spatial frequencies and/or scene complexity. Another set of descriptive parameters may comprise intermediate representations of visual content of the visual neuromodulatory codes, in which case the intermediate representations may be produced by processing the visual neuromodulatory codes using a convolutional neural network trained to perform object recognition and encoding of visual information.

The system 1500 includes one or more sensors 1540, such as biomedical sensors, to measure physiological responses of the subject while the visual neuromodulatory codes are being presented to the subject 1530. For example, the system may include a wristband 1545 and a head-worn apparatus 1547 and may also include various other types of physiological and neurological feedback devices. In general, biomedical sensors include physical sensors, chemical sensors, and biological sensors. Physical sensors may be used to measure and monitor physiologic properties such as, for example, physical blood pressure, respiration, pulse, body temperature, heart sound, respiratory rate, blood viscosity, flow rate, flow rate, etc. Chemical sensors may be utilized to measure chemical parameters, such as, for example, oxygen and carbon dioxide concentration in the human metabolism, pH value, and ion levels in bodily fluids. Biological sensors (i.e., “biosensors”) are used to detect biological parameters, such as tissues, cells, enzymes, antigens, antibodies, receptors, hormones, cholic acid, acetylcholine, serotonin, DNA and RNA, and other proteins and biomarkers.

As noted above, the sensors 1540 used in the system 1500 may include wearable devices, such as, for example, wristbands 1545 and head-worn apparatuses 1547. Other examples of wearable devices include smart glasses, watches, fitness bands/watches, running shoes, rings, armbands, belts, helmets, buttons, etc. In implementations, the physiological responses of the subject may be measured using sensors adapted to measure, inter alia, one of the following: neurological responses, physiological responses, and behavioral responses. The sensors 1540 may include one or more of the following: EEG, MEG, fMRI, ECG, EMG, electrocardiogram, pulse rate, and blood pressure.

The computer subsystem 1505 receives and processes the physiological responses of the subject 1530 measured by the sensors 1540. Specifically, the measured physiological responses and the computed descriptive parameters (of the selected set of descriptive parameters) are input to an algorithm, e.g., an adaptive algorithm 1550, to produce adapted rendering parameters. The system 1500 iteratively repeats the rendering (e.g., by the renderer 1520), computing of descriptive parameters (e.g., by the descriptive parameters calculator 1535), presenting the visual neuromodulatory codes to the subject (e.g., by the display 1525), and processing (e.g., by the adaptive algorithm 1550), using the adapted rendering parameters, until the physiological responses of the subject meet defined criteria. In each iteration, the system 1500 generates one or more adapted visual neuromodulatory codes based on the adapted rendering parameters.

In implementations, the processing of the measured physiological responses of the subject is performed in real time with respect to presenting the visual neuromodulatory codes to a subject while measuring physiological responses of the subject. Alternatively, the processing of the measured physiological responses of the subject may be performed asynchronously with respect to presenting the visual neuromodulatory codes. For example, the measured physiological response data may be stored and processed in batches.

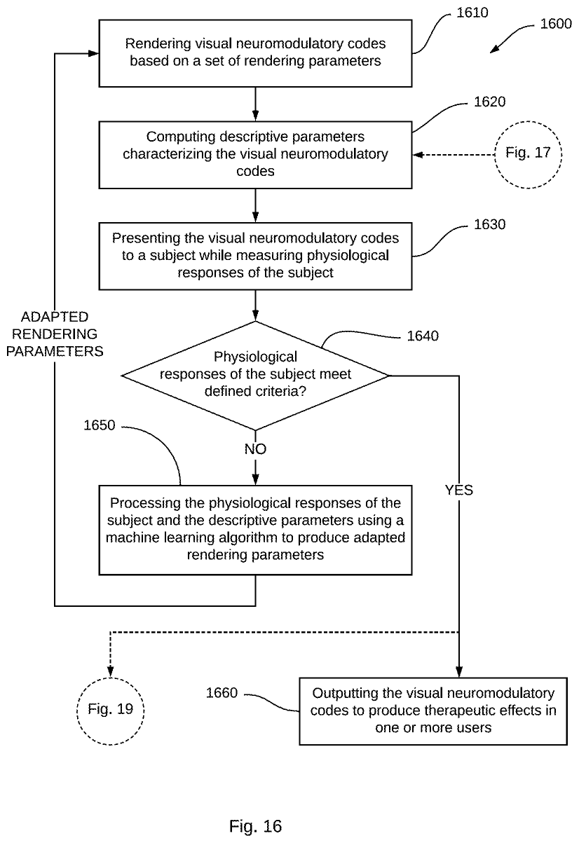

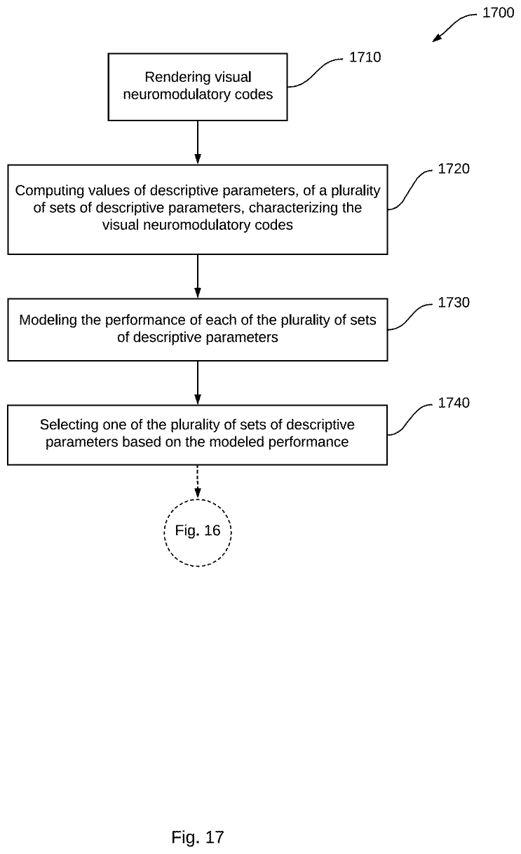

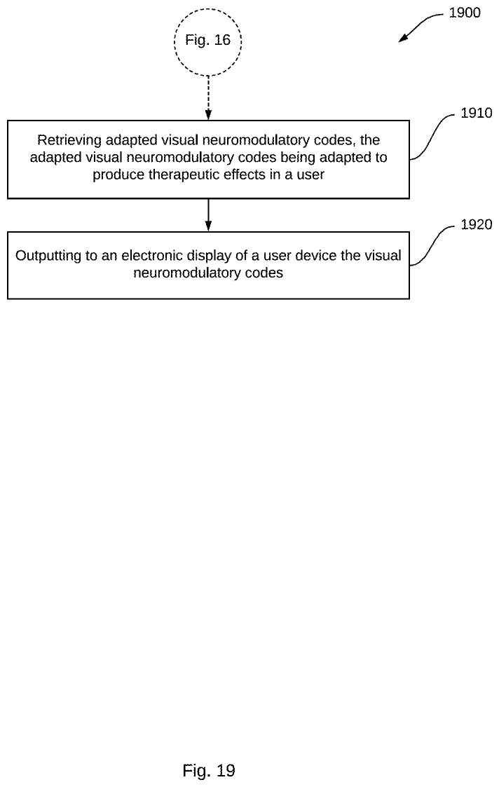

FIG. 16 depicts an embodiment of a method 1600, usable with the system of FIG. 15, to generate visual neuromodulatory codes with a closed-loop approach using an optimized descriptive space. The method 1600 includes rendering visual neuromodulatory codes based on a set of rendering parameters (1610). A set of descriptive parameters is computed characterizing the visual neuromodulatory codes (1620). In implementations, the set of descriptive parameters may be the result of a method to determine a set of optimized descriptive parameters (see, e.g., FIG. 17 and related discussion below). The visual neuromodulatory codes are presented to a subject while measuring physiological responses of the subject (1630). A determination is made as to whether the physiological responses of the subject meet defined criteria (1640). If it is determined that the physiological responses of the subject do not meet the defined criteria, then the physiological responses of the subject and the set of descriptive parameters are processed using a machine learning algorithm to produce adapted rendering parameters (1650). The rendering (1610), the computing (1620), the presenting (1630), and the determining (1640) are repeated using the adapted rendering parameters. If, on the other hand, it is determined that the physiological responses of the subject meet the defined criteria, then the one or more adapted visual neuromodulatory codes are output to be used in producing physiological responses having therapeutic or performance-enhancing effects (1660). For example, in implementations, the adapted visual neuromodulatory codes may be used in a method to deliver visual neuromodulatory codes (see, e.g., FIG. 19 and related description below).

FIG. 17 depicts an embodiment of a method 1700 to determine an optimized descriptive space to characterize visual neuromodulatory codes. The method 1700 includes rendering visual neuromodulatory codes (1710). Values of descriptive parameters (of a plurality of sets of descriptive parameters) are computed characterizing the visual neuromodulatory codes (1720). The performance of each of the sets of descriptive parameters is modeled (1730). One of the sets of descriptive parameters is selected based on the modeled performance (1740).

FIG. 18 depicts an embodiment of a system 1800 to deliver visual neuromodulatory codes generated with a closed-loop approach using an optimized descriptive space. The system 1800 includes an electronic device, referred to herein as a user device 1810, such as mobile device (e.g., mobile phone or tablet) or a virtual reality headset. When symptoms arise, a patient views the visual neuromodulatory codes on a user device, e.g., a smartphone or tablet, using an app or by streaming from a website. In disclosed embodiments, the app or web-based software may provide for the therapeutic visual neuromodulatory codes to be merged with (e.g., overlaid on) content being displayed on the screen, e.g., a website being displayed by a browser, a user interface of an app, or the user interface of the device itself, without interfering with normal use of such content. Audible stimuli may also be produced by the user device in conjunction, or separately from, the visual neuromodulatory codes.

In disclosed embodiments, the system may be adapted to personalize the visual neuromodulatory codes through the use of sensors and data from the user device (e.g., smartphone). For example, the user device may provide for measurement of voice stress levels based on speech received via a microphone of the user device, using an app or browser-based software and, in some cases, accessing a server and/or remote web services. The user device may also detect movement based on data from an accelerometer of the device. Eye-tracking, and pupil dilation measurement, may be performed using a camera of the user device. Furthermore, the user device may present questionnaires to a patent, developed using artificial intelligence, to automatically individualize the visual neuromodulatory codes and exposure time for optimal therapeutic effect. For enhanced effect, patients may opt to use a small neurofeedback wearable to permit further personalization of the visual neuromodulatory codes.

The user device 1810 comprises at least one processor 1815 and memory 1420 (e.g., random access memory, read-only memory, flash memory, etc.). The memory 1820 includes a non-transitory processor-readable medium adapted to store processor-executable instructions which, when executed by the processor 1815, cause the processor 1815 to perform a method to deliver the visual neuromodulatory codes. The user device 1810 has an electronic display 1825 adapted to display images rendered and output by the processor 1815.

The user device 1810 also has a network interface 1830, which may be implemented as a hardware and/or software-based component, including wireless network communication capability, e.g., Wi-Fi or cellular network. The network interface 1830 is used to retrieve one or more adapted visual neuromodulatory codes, which are adapted to produce physiological responses having therapeutic or performance-enhancing effects 1835. In some cases, visual neuromodulatory codes may be retrieved in advance and stored in the memory 1820 of the user device 1810.

In implementations, the retrieval, e.g., via the network interface 1830, of the adapted visual neuromodulatory codes may include communication via a network, e.g., a wireless network 1840, with a server 1845 which is configured as a computing platform having one or more processors, and memory to store data and program instructions to be executed by the one or more processors (the internal components of the server are not shown). The server 1845, like the user device 1810, includes a network interface, which may be implemented as a hardware and/or software-based component, such as a network interface controller or card (NIC), a local area network (LAN) adapter, or a physical network interface, etc. In implementations, the server 1845 may provide a user interface for interacting with and controlling the retrieval of the visual neuromodulatory codes.

The processor 1815 outputs, to the display 1825, visual neuromodulatory codes adapted to produce physiological responses having therapeutic or performance-enhancing effects in a user 1835 viewing the display 1825. The visual neuromodulatory codes may be generated by any of the methods disclosed herein. In this manner, the visual neuromodulatory codes are presented to the user 1835 so that the therapeutic or performance-enhancing effects can be realized. In outputting the adapted visual neuromodulatory codes to the display 1825 of the user device 1810, each displayed visual neuromodulatory code, or sequence of visual neuromodulatory codes (i.e., visual neuromodulatory codes displayed in a determined order), may be displayed for a determined time. These features provide, in effect, the capability of establishing a “dose” which can be prescribed for the user on an individualized basis, in a manner analogous to a prescription medication. In implementations, the determined display time of the adapted visual neuromodulatory codes may be adapted based on user feedback data indicative of responses of the user 1835. In implementations, outputting the adapted visual neuromodulatory codes may include overlaying the visual neuromodulatory codes on displayed content, such as, for example, the displayed output of an app running on the user device, the displayed output of a browser running on the user device 1810, and the user interface of the user device 1810.

The user device 1810 also has a near-field communication interface 1850, e.g., Bluetooth, to communicate with devices in the vicinity of the user device 1810, such as, for example, sensors (e.g., 1860), such as biomedical sensors, to measure physiological responses of the subject 1835 while the visual neuromodulatory codes are being presented to the subject 1835. In implementations, the sensors (e.g., 1860) may include wearable devices such as, for example, a wristband 1860 or head-worn apparatus (not shown). In implementations, the sensors may include components of the user device 1810 itself, which may obtain feedback data by, e.g., measuring voice stress levels, detecting movement, tracking eye movement, and receiving input to displayed prompts.

FIG. 19 depicts an embodiment of a method 1900, usable with the system of FIG. 18, to deliver visual neuromodulatory codes generated with a closed-loop approach using an optimized descriptive space. The method 1900 includes retrieving adapted visual neuromodulatory codes, which are adapted to produce physiological responses having therapeutic or performance-enhancing effects (1910). The method 1900 further includes outputting to an electronic display of a user device the adapted visual neuromodulatory codes (1920). In implementations, the one or more adapted visual neuromodulatory codes may be generated, for example, according to the method of FIG. 16, discussed above.

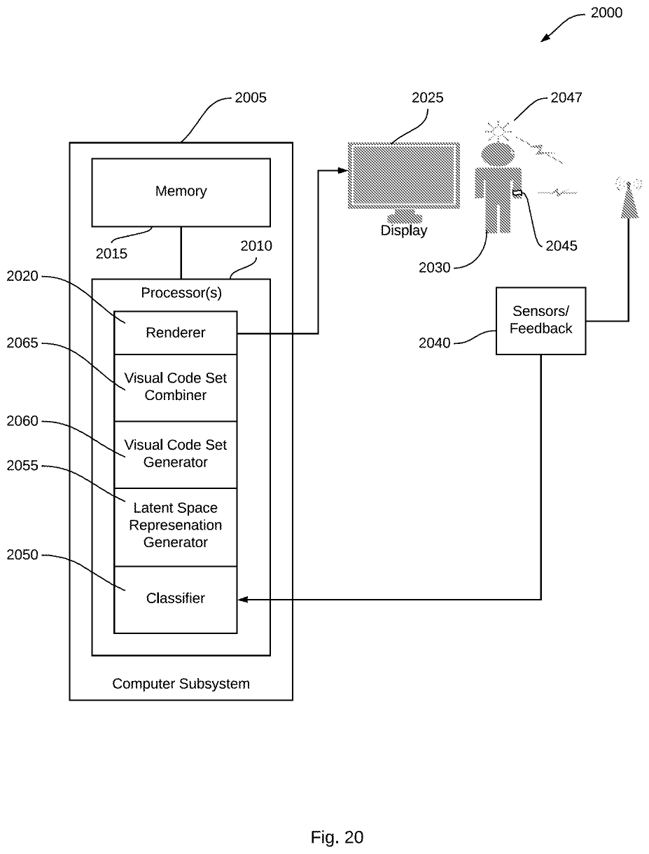

FIG. 20 depicts an embodiment of a system 2000 to generate visual neuromodulatory codes by reverse correlation and stimuli classification. The system 2000 includes a computer subsystem 2005 comprising at least one processor 2010 and memory 2015 (e.g., non-transitory processor-readable medium). The memory 2015 stores processor-executable instructions which, when executed by the at least one processor 2010, cause the at least one processor 2010 to perform a method to generate the visual neuromodulatory codes. Specific aspects of the method performed by the processor are depicted as elements (e.g., code, software modules, and/or processes) within the processor for purposes of discussion only.

The renderer 2020 produces images (e.g., sequences of images) to be displayed on the display 2025 by generating video data based on specific inputs. For example, the renderer 2020 may produce one or more visual neuromodulatory codes (e.g., a sequence of visual neuromodulatory codes) based on an initial set of rendering parameters stored in the memory 2015. The video data and/or signal resulting from the rendering is output by the computer subsystem 2005 to the display 2025.

The system 2000 is configured to present the visual neuromodulatory codes to a subject 2030 by, for example, displaying the visual neuromodulatory codes on a display 2025 arranged so that it can be viewed by the subject 2030. For example, a video monitor may be provided in a location where it can be accessed by the subject 2030, e.g., a location where other components of the system are located. Alternatively, the video data may be transmitted via a network to be displayed on a video monitor or mobile device of the subject. In implementations, the subject 2030 may be one of the users of the system.

In implementations, the system 2000 may present on the display 2025 a dynamic visual neuromodulatory code based on visual neuromodulatory codes. For example, a dynamic visual neuromodulatory code may be formed by combining a number of visual neuromodulatory codes to form a sequence of visual neuromodulatory codes. In a further example, a dynamic visual neuromodulatory code may be adapted to produce at least one of the following effects: a pulsating effect, a zooming effect, a flickering effect, and a color-shift effect. In some cases, the formation of the dynamic visual neuromodulatory code may include processing a set, e.g., a sequence, of visual neuromodulatory codes to produce intermediate images in the sequence of visual neuromodulatory codes. Various techniques, such as interpolation of pixels and gaussian averaging, may be used to produce the intermediate images.

The system 2000 includes one or more sensors 2040, such as biomedical sensors, to measure physiological responses of the subject while the visual neuromodulatory codes are being presented to the subject 2030. For example, the system may include a wristband 2045 and a head-worn apparatus 2047 and may also include various other types of physiological and neurological feedback devices. Other examples of wearable devices include smart glasses, watches, fitness bands/watches, running shoes, rings, armbands, belts, helmets, buttons, etc. In implementations, the physiological responses of the subject may be measured using sensors adapted to measure, inter alia, one of the following: neurological responses, physiological responses, and behavioral responses. The sensors 2040 may include one or more of the following: EEG, MEG, fMRI, ECG, EMG, electrocardiogram, pulse rate, and blood pressure.

The computer subsystem 2005 receives and processes feedback data from the sensors 2040, e.g., the measured physiological responses of the subject 2030. For example, a classifier 2050 receives feedback data while a first set of visual neuromodulatory codes is presented to a subject 2030 and classifies the first set of visual neuromodulatory codes into classes based on the physiological responses of the subject 2030 measured by the sensors 2040. A latent space representation generator 2055 is configured to generate a latent space representation (e.g., using a convolutional neural network) of visual neuromodulatory codes in at least one specified class. A visual neuromodulatory code set generator 2060 is configured to generate a second set of visual neuromodulatory codes based on the latent space representation of the visual neuromodulatory codes in the specified class. A visual neuromodulatory code set combiner 2065 is configured to incorporate the second set of visual neuromodulatory codes into a third set of visual neuromodulatory codes.

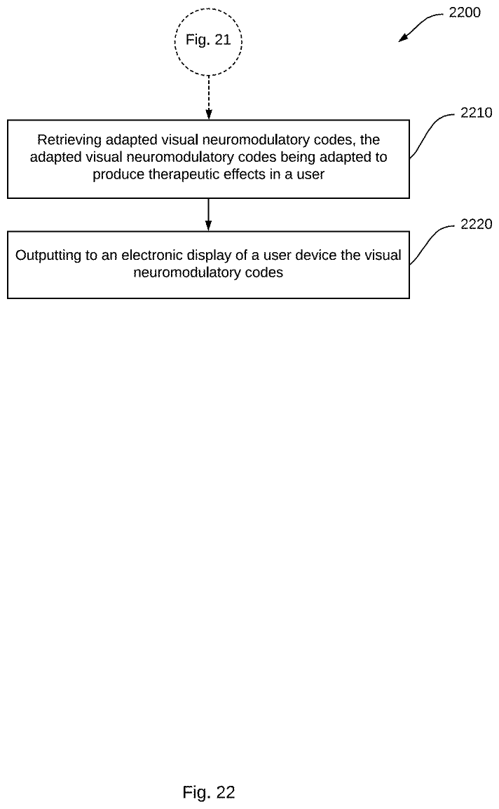

The system 2000 iteratively repeats, using the third set of visual neuromodulatory codes, the classifying the visual neuromodulatory codes, generating the latent space representation, generating the second set of visual neuromodulatory codes, and the combining until a defined condition is achieved. Specifically, the iterations continue until a change in the latent space representation of the visual neuromodulatory codes in specified class, from one iteration to a next iteration, meets defined criteria. The system then outputs the third set of visual neuromodulatory codes to be used in producing physiological responses having therapeutic or performance-enhancing effects. For example, in implementations, the adapted visual neuromodulatory codes may be used in a method to deliver visual neuromodulatory codes (see, e.g., FIG. 22 and related description below). In implementations, the subject 2030 may be one of the users of the system.

In implementations, at least a portion of the first set of visual neuromodulatory codes may be generated randomly. Furthermore, the classifying of the first set of visual neuromodulatory codes into classes based on the measured physiological responses of the subject may include detecting irregularities in the time domain and/or time-frequency domain of the measured physiological responses of the subject 2040.

In implementations, the processing of the measured physiological responses of the subject is performed in real time with respect to presenting the visual neuromodulatory codes to a subject while measuring physiological responses of the subject. Alternatively, the processing of the measured physiological responses of the subject may be performed asynchronously with respect to presenting the visual neuromodulatory codes. For example, the measured physiological response data may be stored and processed in batches.

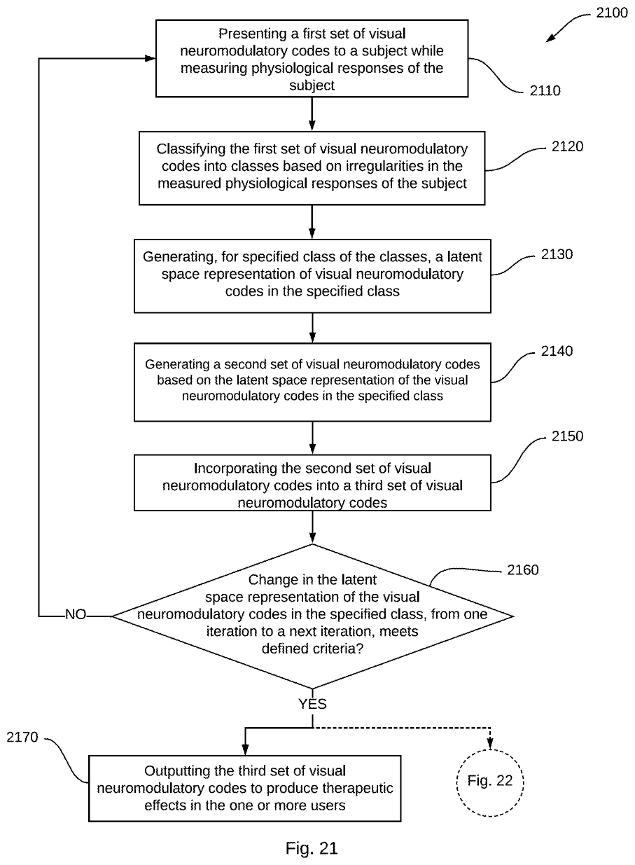

FIG. 21 depicts an embodiment of a method 2100, usable with the system of FIG. 20 to generate visual neuromodulatory codes by reverse correlation and stimuli classification. The method 2100 includes presenting a first set of visual neuromodulatory codes to a subject while measuring physiological responses of the subject (2110). The first set of visual neuromodulatory codes is classified into classes based on the measured physiological responses of the subject (2120). For at least one specified class of the classes, a latent space representation is generated of visual neuromodulatory codes (2130). A second set of visual neuromodulatory codes is generated based on the latent space representation of the visual neuromodulatory codes in the specified class (2140). The second set of visual neuromodulatory codes is incorporated into a third set of visual neuromodulatory codes (2150). If it is determined that a change in the latent space representation of the visual neuromodulatory codes in the at least one specified class, from one iteration to a next iteration, does not meet defined criteria (2160), then the classifying the visual neuromodulatory codes (2120), generating the latent space representation (2130), generating the second set of visual neuromodulatory codes (2140), and the combining (2150) are iteratively repeated using the third set of visual neuromodulatory codes. If the change in the latent space representation of the visual neuromodulatory codes in the at least one specified class, from one iteration to a next iteration, is determined to meet defined criteria (2160), then the third set of visual neuromodulatory codes are output to be used in producing physiological responses having therapeutic or performance-enhancing effects (2170). In implementations, the third set of visual neuromodulatory codes may be used in a method to deliver visual neuromodulatory codes generated by reverse correlation and stimuli classification (see FIG. 22 and related description below).

FIG. 22 depicts an embodiment of a method 2200, usable with the system of FIG. 18, to deliver visual neuromodulatory codes generated by reverse correlation and stimuli classification. The method 2200 includes retrieving one or more adapted visual neuromodulatory codes, the one or more adapted visual neuromodulatory codes being adapted to produce physiological responses having therapeutic or performance-enhancing effects (2210). The method 2200 further includes outputting to an electronic display of a user device the one or more adapted visual neuromodulatory codes (2220). In implementations, the one or more adapted visual neuromodulatory codes may be generated, for example, according to the method of FIG. 21, discussed above.

The foregoing detailed description has set forth various implementations of the devices and/or processes via the use of block diagrams, schematics, and examples. Insofar as such block diagrams, schematics, and examples contain one or more functions and/or operations, it will be understood by those skilled in the art that each function and/or operation within such block diagrams, flowcharts, or examples can be implemented, individually and/or collectively, by a wide range of hardware, software, firmware, or virtually any combination thereof. Those of skill in the art will recognize that many of the methods or algorithms set out herein may employ additional acts, may omit some acts, and/or may execute acts in a different order than specified. The various implementations described above can be combined to provide further implementations.

These and other changes can be made to the implementations in light of the above-detailed description. In general, in the following claims, the terms used should not be construed to limit the claims to the specific implementations disclosed in the specification and the claims, but should be construed to include all possible implementations along with the full scope of equivalents to which such claims are entitled. Accordingly, the claims are not limited by the disclosure.

1. A method for providing spatiotemporal sensory inputs to one or more participants to produce a stimulus map of the brain, the method comprising:
sampling a spatiotemporal sensory code generation model with a first encoding vector to produce a first spatiotemporal sensory code in the form of a first video sequence;
outputting the first video sequence to provide a first spatiotemporal sensory input to said one or more participants;
receiving one or more neural response measurements for said one or more participants, said one or more neural response measurements being performed while the first spatiotemporal sensory input is being presented to each respective one of said one or more participants;
determining an outcome function based, at least in part, on said one or more neural response measurements for said one or more participants;
producing a second encoding vector based, at least in part, on the first encoding vector and the outcome function; and
iteratively repeating said sampling, said outputting, said receiving, and said determining with the second encoding vector, and any successive encoding vectors, until a defined set of stopping criteria for the outcome function is satisfied,
wherein, upon satisfying the defined set of stopping criteria for the outcome function, a resulting spatiotemporal sensory code is stored to form part of a stimulus map of the brain.

2. (canceled)

3. The method of claim 1, wherein the generation model comprises procedural graphics using input parameters including one or more of: spatial frequencies, temporal frequencies, spatial locations, spatial extents, and translation-based motion vectors.

4. The method of claim 1, wherein the spatiotemporal sensory code generation model comprises a generative adversarial network or deep diffusion model and the first encoding vector points to a location in a latent generation space.

5. (canceled)

6. The method of claim 1, wherein the first video sequence has N frames starting from time Tᵢ, and the method further comprises: applying a per-frame window function to the first video sequence; and adding the result to an output frame buffer, filling frames from Tᵢ to Tᵢ+N.

7. The method of claim 1, wherein successive encoding vectors are produced based at least in part on the outcome function and a plurality of preceding encoding vectors.

8. The method of claim 1, wherein said producing the second encoding vector is done at time Tᵢ+S, where S<=N, and the method further comprises: applying the per-frame window function to the second video sequence; and adding the result to the output frame buffer, resulting in the output frame buffer comprising frames Tᵢ to Tᵢ+S+N.

9. The method of claim 8, wherein, during said outputting, frames from Tᵢ to Tᵢ+S are output from the output frame buffer to be presented to said one or more participants while the second video sequence is being produced.

10. The method of claim 1, wherein said outputting comprises displaying said sequence of spatiotemporal sensory inputs to one or more electronic screens.

11. (canceled)

12. The method of claim 1, wherein said one or more neural response measurements are received from a multiple-channel buffer comprising current multiple-channel neural response measurements and previous multiple-channel neural response measurements.

13. The method of claim 1, further comprising:
aligning timewise, across said one or more participants, said one or more neural response measurements;
extracting one or more features for each measurement time step across said one or more neural response measurements and across said one or more participants; and
comparing said one or more extracted features to targets to calculate the outcome function.

14. (canceled)

15. The method of claim 1, wherein, in storing said resulting spatiotemporal sensory code to form part of the stimulus map of the brain, a feature representation of said one or more neural response measurements is associated with a location in a high dimensional space.

16. The method of claim 1, said resulting spatiotemporal sensory code is associated with a neural state at a specific brain location.

17. The method of claim 1, said resulting spatiotemporal sensory code is associated with a whole-brain neural state which is defined in terms of multivariate cross-coherence across spectral bands and said resulting spatiotemporal sensory code is adapted to maximize the cross-coherence across one or more pairs of nodes of the brain map.

18. (canceled)

19. (canceled)

20. A system for providing spatiotemporal sensory inputs to one or more participants to produce a stimulus map of the brain, the system comprising:
one or more processors in communication with a memory, the memory storing instructions executable by said one or more processors to perform:
sampling a spatiotemporal sensory code generation model with a first encoding vector to produce a first spatiotemporal sensory code in the form of a first video sequence;
outputting the first video sequence to provide a first spatiotemporal sensory input to said one or more participants;
receiving one or more neural response measurements for said one or more participants, said one or more neural response measurements being performed while the first spatiotemporal sensory input is being presented to each respective one of said one or more participants;
determining an outcome function based, at least in part, on said one or more neural response measurements for said one or more participants;
producing a second encoding vector based, at least in part, on the first encoding vector and the outcome function; and
iteratively repeating said sampling, said outputting, said receiving, and said determining with the second encoding vector, and any successive encoding vectors, until a defined set of stopping criteria for the outcome function is satisfied,
wherein, upon satisfying the defined set of stopping criteria for the outcome function, a resulting spatiotemporal sensory code is stored to form part of a stimulus map of the brain.

21. The system of claim 20, wherein the first video sequence has N frames starting from time Tᵢ, and the method further comprises: applying a per-frame window function to the first video sequence; and adding the result to an output frame buffer, filling frames from Tᵢ to Tᵢ+N.

22. The system of claim 20, wherein said producing the second encoding vector is done at time Tᵢ+S, where S<=N, and the method further comprises: applying the per-frame window function to the second video sequence; and adding the result to the output frame buffer, resulting in the output frame buffer comprising frames Tᵢ to Tᵢ+S+N.

23. The system of claim 20, wherein said outputting comprises displaying said sequence of spatiotemporal sensory inputs to said one or more electronic screens.

24. The system of claim 20, wherein said one or more neural response measurements are received from a multiple-channel buffer comprising current multiple-channel neural response measurements and previous multiple-channel neural response measurements.

25. The system of claim 20, wherein the memory further stores instructions executable by said one or more processors to perform:
aligning timewise, across said one or more participants, said one or more neural response measurements;
extracting one or more features for each measurement time step across said one or more neural response measurements and across said one or more participants; and
comparing said one or more extracted features to targets to calculate the outcome function.

26. A non-transitory computer-readable medium storing instructions that, when executed by one or more processors, cause said one or more processors to perform a method for providing spatiotemporal sensory inputs to one or more participants to produce a stimulus map of the brain, the method comprising:
sampling a spatiotemporal sensory code generation model with a first encoding vector to produce a first spatiotemporal sensory code in the form of a first video sequence;
outputting the first video sequence to provide a first spatiotemporal sensory input to said one or more participants;
receiving one or more neural response measurements for said one or more participants, said one or more neural response measurements being performed while the first spatiotemporal sensory input is being presented to each respective one of said one or more participants;
determining an outcome function based, at least in part, on said one or more neural response measurements for said one or more participants;
producing a second encoding vector based, at least in part, on the first encoding vector and the outcome function; and
iteratively repeating said sampling, said outputting, said receiving, and said determining with the second encoding vector, and any successive encoding vectors, until a defined set of stopping criteria for the outcome function is satisfied,
wherein, upon satisfying the defined set of stopping criteria for the outcome function, a resulting spatiotemporal sensory code is stored to form part of a stimulus map of the brain.
